(19) United States
(12) Patent Application Publication
(10) Pub. No.: US 2022/0370463 A1
(43) Pub. Date: Nov. 24, 2022

(54) STABILIZED C-FMS INTRACELLULAR FRAGMENTS (FICD) PROMOTE OSTEOCLAST DIFFERENTIATION AND ARTHRITIC BONE EROSION

(71) Applicant: New York Society for the Relief of the Ruptured and Crippled, Maintaining the Hospital for Special S, New York, NY (US)

(72) Inventor: Kyung-Hyun Park-Min, Fort Lee, NJ (US)

(21) Appl. No.: 17/761,027

(22) PCT Filed: Sep. 18, 2020
(86) PCT No.: PCT/US2020/051590

§ 371 Date: Mar. 16, 2022

Related U.S. Application Data
(60) Provisional application No. 62/902,782, filed on Sep. 19, 2019.

(51) Int. Cl. A61K 31/52; A61K 31/4439; G01N 33/68

(52) U.S. Cl. CPC A61K 31/52; A61K 31/4439; G01N 33/6863

(57) ABSTRACT

Provided herein is a method of treating bone resorption associated with osteoclastic activity in a subject in need thereof. The method includes reducing the level of FMS intracellular fragments (FICDs) in the subject.

### STATEMENT REGARDING FEDERALLY SPONSORED RESEARCH OR DEVELOPEMNT

This invention was made with government support under AR061430 and AR069562 awarded by the National Institutes of Health. The government has certain rights in the invention.

### BACKGROUND

Rheumatoid arthritis (RA) is a chronic inflammatory and autoimmune disorder (Smolen, J. S. et al. Rheumatoid arthritis. Nat Rev Dis Primers 4, 18001, doi:10.1038/nrdp.2018.1 (2018)). Bone erosion is one of the key clinical features of RA and is closely linked to impaired mobility of patients with RA (Schett, G. & Gravallese, E. Bone erosion in rheumatoid arthritis: mechanisms, diagnosis and treatment. Nat Rev Rheumatol 8, 656-664, doi:10.1038/nrrheum.2012.153 (2012)). However, the underlying mechanisms of arthritic bone erosion by osteoclasts have not been fully determined (Guo, Q. et al. Rheumatoid arthritis: pathological mechanisms and modern pharmacologic therapies. Bone Res 6, 15, doi:10.1038/s41413-018-0016-9 (2018)). In addition to inflammatory cytokines such as TNF-alpha, M-CSF and its receptor c-FMS have also been implicated in the pathogenesis of RA and arthritic bone erosion (Lin, H. et al. Discovery of a cytokine and its receptor by functional screening of the extracellular proteome. Science 320, 807-811, doi:10.1126/science.1154370 (2008)). In patients with RA, the level of M-CSF increases in the serum and synovial fluid (Paniagua, R. T. et al. c-Fms-mediated differentiation and priming of monocyte lineage cells play a central role in autoimmune arthritis. Arthritis Res Ther 12, R32, doi:10.1186/ar2940 (2010)), and inhibition of c-FMS activation attenuates the progression of joint inflammation and bone erosion in animal models of arthritis (Ohno, H. et al. The orally-active and selective c-Fms tyrosine kinase inhibitor Ki20227 inhibits disease progression in a collagen-induced arthritis mouse model. Eur J Immunol 38, 283-291, doi:10.1002/eji.200737199 (2008)). Despite the importance of M-CSF in the differentiation of myeloid cells (Pollard, J. W. Trophic macrophages in development and disease. Nat Rev Immunol 9, 259-270, doi:10.1038/nri2528 (2009)), very little is known about the molecular mechanism underlying the role of M-CSF/c-FMS in arthritic bone erosion.

New diagnostic markers and treatment for RA and other diseases associated with osteoclastic bone resorption are needed.

### SUMMARY OF THE INVENTION

Provided herein, in a first aspect, is a method of treating bone resorption associated with osteoclastic activity in a subject in need thereof. The method includes reducing the level of FMS intracellular fragments (FICDs) in the subject. In one embodiment, the method includes administering an inhibitor of MNK1/2. In another embodiment, the method includes administering an inhibitor of calpain 1 or pan-Calpain inhibitor. In yet another embodiment, the method includes inhibiting TNF-alpha converting enzyme (TACE).

In another aspect, a method of diagnosing and treating bone loss associated with osteoclastic activity in a subject is provided. The method includes (i) quantifying the amount of FMS intracellular fragments (FICDs) in a sample from the subject; and/or (ii) quantifying the amount of circulating soluble c-FMS in a sample from the subject; and (iii) diagnosing a bone loss in the subject when an increase in FICDs or soluble c-FMS is detected as compared to a control. The method includes treating the subject for the bone loss.

In another aspect, a method of assessing the efficacy of a treatment for a bone loss is provided. The method includes (i) quantifying the amount of FMS intracellular fragments (FICDs) in a sample from the subject; and/or (ii) quantifying the amount of circulating soluble c-FMS in a sample from the subject; and (iii) wherein a decrease in the amount of FICDs or soluble c-FMS as compared to a control indicates the treatment is at least partially efficacious.

Other aspects and advantages of the invention will be readily apparent from the following detailed description of the invention.

### DESCRIPTION OF THE FIGURES

FIGS. 1A-1J show the detection of small fragments of c-FMS and soluble c-FMS. (a) Immunoblot of RA synovial CD14+ cells with antibodies against C-terminal of c-FMS. (b) Human CD14+ cells from healthy donors were cultured with M-CSF at the indicated times. Immunoblot of whole cell lysates with antibodies against C-terminal of c-FMS. (c) Immunoblot of whole cell lysates from CD14+ cells from healthy, RA, and OA. (d and e) Human CD14+ cells were nucleofected by control (CTL) or TACE siRNAs and then were cultured with M-CSF. (d) Efficiency of knock down. TACE mRNA was measured by qPCR and normalized by HPRT. (e) Immunoblot of FICD using anti-c-FMS antibody. (f and g) Soluble c-FMS in synovial fluids from patients with rheumatoid arthritis (RA, n=13) and osteoarthritis (OA, n=8) were measured by ELISA (f) and by immunoblot with antibodies against N-terminal c-FMS (g). (h and i) Human CD14+ cells were cultured with M-CSF. A soluble c-FMS in culture media was measured with ELISA (h) and immunoblot by anti-c-FMS antibodies (i). All data are shown as mean±SEM. *, p<0.05 by unpaired t-test (d,e) and One-way ANOVA with a post hoc Tukey test (f). Data represent at least 3 independent donors. M; a mature c-FMS, I; an immature c-FMS, #; small fragments. FIG. 1J shows the cleavage of c-FMS by TACE resulting in FICDs.

FIGS. 2A-2G demonstrate that calpain 1 cleaves FICDs in the nucleus. (a) Human CD14+ cells were cultured with M-CSF (20 ng/ml) for 8 hours to induce early signals and then DAPT (10uM) was added for 2 days. Protein expression of c-FMS, Na+K+ pump, Lamin B1 and α-tubulin, as determined by immunoblot. ME; membrane extracts, CE; cytoplasmic extracts, and NE; nuclear extracts. (b) Immunohistochemistry of DAPI and c-FMS [middle]. Right panel shows a merged image. Scale: 200×. (c) Cells were starved for three hours and then stimulated with M-CSF for the indicated times. (d and e) Cells were treated with Imatinib (0.3 μM, d) or c-FMS blocking antibody (5 μg) prior to the addition of M-CSF. Protein expression of FICD was measured by immunoblot. Lamin B1 and α-tubulin were used as controls for nuclear and cytoplasmic fractions, respectively. (f) Human CD14+ cells were cultured with M-CSF (20 ng/ml) for 8 hours to induce early signals and then MDL 28170(5 uM). Immunoblot with anti c-FMS and Lamin B1 antibodies. (g and h) Calpain 1, 5, and 6 were knocked down with siRNAs. Cells were cultured with M-CSF for 12 hours. (g) Efficiency of knockdown of Calpain 1, 5, and 6. (h) Immunoblot of c-FMS and Lamin B1. All data are shown as mean±SEM. *,p<0.05 by two-tailed, unpaired t-test (g). Representative results from at least three independent experiments.

FIGS. 3A-3G demonstrate that c-FMS proteolysis positively regulates osteoclastogenesis. (a) Schematic showing mutations in the TACE cleavage sites of c-FMS. TACE cleavage sites of c-FMS were replaced by addition of 14 amino acids from insulin receptor sequences (FMS^{mut}). (b) 293T cells did not express c-FMS and were transduced by lentiviral particles encoding control, FMS^{wt} or FMS^{mut}. Cells were then stimulated with M-CSF for the indicated times. Protein expression of phosphor-ERK, phospho-JNK, phospho-p38, and α-tubulin was determined by immunolbot. (c-f) BMDMs from Csf1r^{f/+} Mx1-Cre mice were transduced with lentivirus encoding control, wild type FMS (FMS^{wt}), or TACE-uncleavable mutant FMS (FMS^{mut}). Transduced BMDMs were stimulated with LPS (10 ng/ml) for 3 hrs (c) and 24 hrs (d). (C) mRNA expression of TNFα and IL6 mRNA was measured by q-PCR. (d) TNFα and IL6 in the culture media were measured by Luminex multiplex cytokine assay. (e) Osteoclastogenesis assay. Left panel shows representative images of TRAP-stained cells. Right panel shows the percentage of TRAP-positive multinuclear cells (MNCs: more than three nuclei) per control from six independent experiments. (n=6) (f) Resorption pit assay. Bone resorption activity analysis of FMS^{con}, FMS^{wt}, or FMS^{mut} cells. Left panel shows representative images and right panel shows the percentage of resorbed pit area per total area. Black scale bar is 100 μm and red scale bar is 200 μm. All data are shown as mean±SEM. *, p<0.05 by One-way ANOVA with a post hoc Tukey test (c-g). Data represent at least three experiments (b-d, g).

FIG. 4A-4I show that FICDtg^{M} mice exhibit osteoporotic bone phenotype with increased osteoclast numbers. (a-c) Bone marrow derived OCPs were transduced by retrovirus encoding either control or FICD. (a) The expression of FICD protein was determined by immunoblot. (b) Osteoclastogenesis assay. Left panel shows representative images of TRAP-stained cells (n=4). (c) Bone resorption activity analysis of control and FICD. Left panel shows representative images and Right panel shows the percentage of resorbed pit area per total area (n=3). (d) The expression of FICD protein. (e and f) Micro-CT analysis of femurs from 12-week-old male wild type (WT) and FICDtg^{M} mice (n=7). (e) Representative images. (f) The indicated parameters in distal femurs. Bone volume/tissue volume ratio (BV/TV), trabecular thickness (Tb.Th), trabecular numbers (Tb.N), and trabecular space (Tb. Sp) were determined by micro CT analysis. (g and h) Histomorphometry analysis of the distal femur of 12-week-old male wild type and FICDtg^{M} mice (n=6). (g) Representative image showing TRAP-positive, multinucleated osteoclasts (red arrow). (h) The number of osteoclasts per bone surface (N.Oc/BS), osteoclast surface area per bone surface (Oc.S/BS), and eroded surface per bone surface (ES/BS). (i) CTX-1 (WT=5, FICDtg^{M}=8) and P1NP (n=7) levels in serums from wild type (WT) and FICDtg^{M} mice. All data are shown as mean±SEM. *,p<0.05 by two-tailed, unpaired t-test (b,c,f,h,i).

FIGS. 5A-5H demonstrate that FICDs augment arthritic bone erosion. (a and b) BMDMs from WT and FICDtg^{M} were cultured with M-CSF and RANKL for 3 days. (a) Osteoclastogenesis assay. Left panel shows representative images of TRAP-stained cells. Right panel shows the percentage of TRAP-positive multinuclear cell per WT cells(n=3). (b) Resorption pit assay. Left panel shows representative images and Right panel shows the percentage of resorbed pit area per total area(n=3). BMDMs From WT and FICDtg^{M} mice were stimulated with LPS (10 ng/ml) for 3 hr (c) and 24 hrs (d). (c) mRNA expression of TNFα and IL6 mRNA was measured by q-PCR. (d) TNFα and IL6 proteins in the culture media were measured by Luminex multiplex cytokine assay. (e-h) K/BxN serum transfer induced arthritis model. 8-week old female wild type and FICDtg^{M} mice were received K/BxN serum on day 0 and day 2. (e and f) Time course of joint swelling and clinical score of serum-induced arthritis in littermate control and FICDtg^{M} mice (n=6). (g)Representative images of TRAP-stained tarsal bones (hind paws) of arthritic mice. (h) Histomorphometry analysis of tarsal bones. N.OC/B.Pm; Osteoclast number/bone parameter. OC.S/BS; osteoclast surface/bone surface, and ES/BS; Eroded surface/bone surface. Black scale bar is 100 μm and red scale bar is 200 μm. n.s.: not significant. All data are shown as mean±SEM. *, p<0.05 two-way ANOVA with a post hoc Tukey test (c-f) or two-tailed, unpaired t-test (a,b,h).

FIGS. 6A-6N show that FICD augments NFATc1 expression by activating the MNK1/2/eIF4E axis. (a and b) BMDMs from WT and FICDtg^{M} mice was stimulated with RANKL (50 ng/ml) at the indicated time. (a) RT-qPCR of Nfatc1 mRNA normalized relative to Hprt mRNA. (b) Immunoblot with anti NFATc1, HA, or α-tubulin antibodies. (c and d) BMDMs from Csf1r^{f/+} Mx1-Cre mice were transduced by lentiviruses encoding FMS^{wt}, or FMS^{mut} and then cultured with M-CSF and RANKL. (c) Immunoblot of whole lysate with anti-NFATc1 antibody. α-tubulin was used as a control. Left panel shows the representative images. Right panel shows the intensity of NFATc1 bands. The intensity of NFATc1 in FMS^{mut} was set as 100%. (d) Expression mRNA level of NFATc1. (e) Immunoblot of whole cell lysates with phospho-eIF4E antibodies. HA-tagged FICD was detected by HA-antibody. α-tubulin was used as a control. Left panel shows the representative images. Right panel shows the percentage of intensity of band (at 24 hrs) relative to control from three independent experiments. (f and g) Human CD14+ cells were treated with CGP57380 at the indicated doses for one hour and cultured with RANKL for one day. D: DMSO (f) Immunoblot with anti-NFATc1, phospho-eIF4E, or α-tubulin antibodies Left panel shows the representative images. Right panel shows the percentage of intensity of band relative to control (n=4). (g) Nfatc1 mRNA expression was measured by qPCR relative to HPRT. A DMSO-treated RANKL condition was set as 100%. (h) Osteoclastogenesis assay. BMDMs from WT and FICDtg^{M} mice were treated with CPG57380 at the indicated doses and then cultured with RANKL for three days. Upper panel shows representative images of TRAP-stained cells. Bottom panel shows the percentage of TRAP-positive multinuclear cells (MNCs: more than three nuclei) per control from three independent experiments. Scale bar: 100 μm. (i) Cell viability assay. BMDMs WT and FICDtg^{M} mice was stimulated with CPG57380 at the indicated doses for one day. (j-n) K/BxN serum transfer induced arthritis model. 9-week old male C57BL/6J mice were received K/BxN serum on day 0 and day 2. Vehicle or CPG57380 (40 mg/kg) was administrated intraperitoneally (i.p) at day 2 until day 13 (n=5-6). (j) Schematic diagram showing experimental design. (k) Ankle thickness. (1) Arthritis score. (m) Representative images of TRAP stained histological sections from calcaneocuboid and tarsometatarsal joints. (n) Histomorphometry analysis of tarsal bones. N.OC/B.Pm; Osteoclast number/bone parameter. OC.S/BS; osteoclast surface/bone surface. ES/BS; Eroded surface/bone surface (n=5-6). All data are shown as mean±SEM. *, p<0.05 by One-way ANOVA with a post hoc Tukey test (a, f-i,k,l) or two-tailed, unpaired t-test (d,e,n). Data represent at least three experiments.

FIGS. 7A-7I show that FICDs enhance the activation of MNK1/2/eIF4E via DAP5/Fxr1. (a) Ingenuity Pathway analysis of 145 FICD-interacting proteins. Pooled data from two biological replicates were analyzed. (b) Interaction map showing 20 FICD-interacting proteins in Protein Synthesis pathways by STRING functional protein association analysis. (c) Frequency of proteins shown in (b). (d) Interaction of FICD with DAP5 or Fxr1 was determined by immunoblot analysis by anti-DAP5, Fxr1, HA, or α-tubulin antibodies. Whole cell lysates of BMDMs from WT and FICDtg^{M} mice were used for immunoprecipitation with anti-HA-tag antibodies. (e-i) Knock-down of DAP5 (e and f) or Fxr1 (g and h) in both human CD14+ cells (e, g, and i) and BMDM (f and h). (e-h) Protein expression of NFATc1, p-eIF4E, eIF4E, DAP5, Fxr1 and α-tubulin was determined by immunoblot. (i) Osteoclastogenesis assay. Left panel shows representative images of TRAP-stained cells. Right panel shows the percentage of TRAP-positive multinuclear cells (MNCs: more than three nuclei) per control from three independent experiments. CTL: Control. All data are shown as mean±SEM. *, p<0.05 by one-way ANOVA with a post hoc Tukey test (I). Data represent 2 biological replicates for mass spectrophotometry (a-c) and 3 three independent experiments (d-i).

FIGS. 8A-8I show c-FMS expression in synovial macrophages. (A) The levels of M-CSF in synovial fluids from patients with rheumatoid arthritis (RA, n=13) and osteoarthritis (OA, n=8) were measured. (B) Mass spectrometry analysis for proteins from ˜50 kDa bands (red box) identified c-FMS as one of top genes. An image of coomassie blue stained genes showing immunoprecipitated cells lysates with either anti-DDK-tag antibodies or IgG control. (C-G) Human CD14+ cells were cultured with M-CSF (20ng/ml). Immunoblot of c-FMS with different antibodies against C-terminal of c-FMS; (C) C-20, (D) D-8, and (E) #3152. Immunoblot of c-FMS with different antibodies against N-terminal of c-FMS; (F) #61701, (G) H-300. (H-I) Human CD14+ cells were cultured with M-CSF (20 ng/ml) for 8 hours to induce early signals, and then BB94 (10 uM) was added for 2 days. (H) Immunoblot with anti c-FMS and α-tubulin antibodies in whole lysates (n=3). (I) Soluble c-FMS was detected by ELISA (n=3). The treatment of BB94 inhibited the shedding of c-FMS and diminished soluble c-FMS in the media. All data are shown as mean±SEM. *; p<0.05 significant by two-tailed, paired t-test (A and I). M: mature form, I: immature form, H: hgh mass, L: low mass.

FIGS. 9A-9C show the cellular localization of FICDs. (A) Confocal microscopy of human CD14+ cells labeled with antibodies against C-terminal of c-FMS (middle) and DAPI (Left). White scale bar is 10 μm. (B and C) Human CD14+ cells were cultured with M-CSF for 12 hrs and then was stimulated with IL-34 (20 ng/ml) for the indicated time (C). (B) A schematic diagram illustrating the experiment design for FIG. 9C and FIG. 3D, (C) Immuno-blot of cytoplasmic and nuclear lysate with anti c-FMS, Lamin B1, and α-tubulin antibodies. M: mature c-FMS, I: immature c-FMS, H-FICD: high mass FICD, L-FICD; low mass FICD. Data represent at least three independent experiments.

FIGS. 10A-10F show calpain regulates FICD generation. (A and B) Human CD14+ cells were cultured with M-CSF (20 ng/ml) for 8 hours to induce early signals and then (A) MDL 28170(0, 1, 2, 5 uM) or (B) PD150606 (0, 2, 5 uM) was added for 2 days. Immunoblot with anti c-FMS and α-tubulin antibodies in whole lysates(n=3). (C) Immunoblot of nuclear lysates with c-FMS and Lamin B1 antibodies. Cells were cultured with M-CSF for 12 hours, and then M-CSF was removed. Cells were subsequently treated with or without CaCl2 (10 mM) for an additional 24 hours. (D) Osteoclastogenesis assay. Human CD14+ cells were cultured with M-CSF, RANKL and MDL28170 for 4 days. Left panel shows representative images of TRAP-stained cells. Right panel shows the percentage of TRAP-positive multinuclear cells (MNCs: more than three nuclei) per control (DMSO) from three independent experiments. Black scale bar is 100 μm. (E) A table showing the calpain cleavage site in c-FMS predicted by GPS-CCD program. First two calpain cleavage sites in cytoplasmic domains of c-FMS were used for FICD constructs. (F) A schematic diagram of a FICD construct. Representative results from at least three independent experiments.

FIGS. 11A-11E show calpain regulates FICD generation. (A-E) K/BxN serum transfer induced arthritis model. 8-week old male C57BL/6J mice were received K/BxN serum on day 0 and day 2. Vehicle or MDL28170 (10 mg/kg) was administrated intraperitoneally (i.p) at day 2 until day 11 (n=10). (A) K/BxN Experimental design. (B) Arthritis score. (C) Ankle thickness. (D) Representative images of TRAP stained histological sections from calcaneocuboid and tarsometatarsal joints. (E) Histomorphometry analysis of tarsal bones. N.OC/B.Pm; Osteoclast number/bone parameter. OC.S/BS; osteoclast surface/bone surface. ES/BS; Eroded surface/bone surface (n=7). All data are shown as mean±SEM. *, p<0.05 by two-tailed, unpaired t-test (E) or One-way ANOVA with a post hoc Tukey test (B, C).

FIGS. 12A-12D show c-FMS proteolysis and TACE-cleavage resistant form of c-FMS. (A) 293T cells has no c-FMS expression and were transduced by lentiviruses encoding control, FMSwt, or FMSmut and then stimulated with or without 12-O-Tetradecanoylphorbol-13-acetate (TPA, 100 ng/ml) to activate TACE. The expression of FMSwt and FMSmut was analyzed by flow cytometry. Left panel shows representative images, and right panel shows the accumulative quantification from three independent experiments. (B) Protein expression analysis of c-FMS by immunoblot. BMDMs from FMS+/+MX1 cre and FMSf/+MX1cre mice were cultured with M-CSF (20 ng/ml) for two days. The levels of FICD in FMSf/+MX1cre BMDMs (lane 2) were 20% of FMS+/+MX1cre (lane 1) (C and D) Protein expression analysis of FMSwt and FMSmut by immunoblot. FICD expression was diminished in FMSmut compared to FMSwt in BMDMs (C) or 293T cells (D) Representative results from at least three independent experiments. All data are shown as mean±SEM. *; p<0.05 **; p<0.05, n.s; not significant by two-tailed, paired t-test (A). Arrow: FICD. All data represent at least three experiments.

FIGS. 13A-13C demonstrate generation of FICDKI/KI^{M} mice. (A) Construction of the FICD-HA knock-in (KI) targeting vector. See the Experimental Procedures for details. The gene encoding ROSA26 (WT allele); the targeting vector (targeting vector); poly A (filled gray square); loxP sequence (filled blue triangle); SDA (self-deletion anchor) site (filled black triangle); CAG promoter; Neo cassette; Diphtoxin A gene (DTA); the targeted allele with the targeting vector (target allele); SDA mediated Neo deletion (Conditional KI allele); cre-mediated expression after removal of polyA between CAG promoter and FICD-HA gene (Constitutive KI allele). (B) Southern blot analyses of DNA from WT ES cells or FICDKI ES cells. Genomic DNA was extracted from ES cells, digested with BstEII or EcoRV, and analyzed by Southern blot by Neo probe (red) shown in FIG. A. Southern analysis with Neo probe generates 12.95 kb fragments after digestion with EstEII (upper panel) and 10.95 kb fragments after digestion with EcoRV (lower panel). (C) PCR analyses of DNA from wild type LysM cre (WT), FICDKI/+ LysM cre or FICDKI/KI LysM cre mice. Genomic DNA was extracted from mouse tail tissue. Primers for PCR are shown as arrows. The sequence of primers is listed in Table 1.

FIGS. 14A and 14B show micro-CT analysis of FICDtg^{M} female mice. (A and B) μ-CT analysis of femurs from 12-week-old female wild type (WT, n=6) and FICDtg^{M} mice (n=7). (A) Representative images. (B) Bone parameters in distal femurs. Bone volume/tissue volume ratio (BV/TV), trabecular thickness (Tb.Th), trabecular numbers (Tb.N), and trabecular space (Tb.Sp) were determined by μ-CT analysis. Black scale bar: 100 μm. All data are shown as mean±SEM. *, p<0.05; n.s, not signficant by unpaired t-test (B).

FIGS. 15A-15D show overt phenotype of FICDtg^{M} mice is comparable to wild type. (A and B) The comparison of body weight between wild type (WT) and FICDtg^{M} male (A) and female (B) mice. (C)The comparison of spleen weight between wild type (WT) and FICDtg^{M} male and female mice. (D) The comparison of femur length between wild type (WT) and FICDtg^{M} male and female mice. All data are shown as mean±SEM. n.s., not significant; *, p<0.05 by One-way ANOVA with a post hoc Tukey test or two-tailed, unpaired t-test (A and B) or two-tailed, unpaired t-test (C and D).

FIGS. 16A-D demonstrate FICD deficiency in FMS null background diminished bone mass and increased osteoclast numbers. (A and B) μ-CT analysis of femurs from 12-week-old male FMS cKO (control, n=6) and FMScKOFICDtg^{M} mice (n=7). (A) Representative images. (B) Bone parameters in distal femurs. Bone volume/tissue volume ratio (BV/TV), trabecular thickness (Tb.Th), trabecular numbers (Tb.N), and trabecular space (Tb.Sp) were determined by μ-CT analysis. (C and D) Histomorphometry analysis of the distal femur of 12-week-old male FMS cKO(control, n=4) and FMScKOFICDtg^{M} mice (n=5). (C) Representative images showing TRAP-positive, multinucleated osteoclasts (red arrow). (D) The number of osteoclasts per bone surface (N.Oc/BS), osteoclast surface area per bone surface (Oc.S/BS), and eroded surface per bone surface (ES/BS). Black scale bar is 100 μm and red scale bar is 200 μm. All data are shown as mean±SEM. *, p<0.05 by two-tailed, unpaired t-test (B and D).

FIGS. 17A-17G demonstrate that ablation of Raptor has a minimal effect on NFATc1 protein expression. (A and B) BMDMs from WT and FICDtg^{M} mice were cultured with RANKL for 24 hours. Immunoblot of whole cell lysates with phopho-p70S6K and phopho-4E-BP1 antibodies. HA-tagged FICDs were detected by HA-antibody. α-tubulin or P38 was used as a control. (C) BMDMs from female wild type LysM cre mice (WT) or RAPTORf/f-LysM cre mice (Raptor cKO) were cultured with M-CSF for 4 days and were stimulated with RANKL for the indicated days. Immunoblot of whole cell lysates with anti-Raptor, NFATc1, or α-tubulin antibodies. (D-F) BMDMs from WT mice were treated with CPG57380 at the indicated doses and then cultured with RANKL for one day. (D) Immunoblot of whole lysate with anti-NFATc1 antibody. α-tubulin was used as a control. Left panel shows the representative images. Right panel shows the percentage of intensity of NFATc1 bands (24 h). The intensity of NFATc1 bands in RANKL treatment conditions (control) was set as 100%. (n=4) (E) Expression mRNA level of NFATc1. (F) BMDMs from WT mice were treated with CPG57380 at the indicated doses and then cultured with RANKL for 3 days. D: DMSO. All data are shown as mean±SEM. *, p<0.05 by one-way ANOVA with a post hoc Tukey test (D-F). Data represent at least three experiments (A, B, D-F) and 2 biological replicates (C).

FIG. 18 shows that the DAP5/Fxr1 axis regulates mouse osteoclastogenesis. Osteoclastogenesis assay. DAP5 or Fxr-1 was knocked down with siRNAs. BMDM cells were cultured with M-CSF and RANKL for 3 days. Left panel shows representative images of TRAP-stained cells. Right panel shows the percentage of TRAP-positive multinuclear cells (MNCs: more than three nuclei) per control from three independent experiments. CTL: Control. Black scale bar: 100 μm. All data are shown as mean±SEM. *, p<0.05 by one-way ANOVA with a post hoc Tukey test. Data represent at least three independent experiments.

FIG. 19 shows the proposed model: c-FMS proteolysis cooperates with MNK1/2 pathways to promote RANKL-induced osteoclastogenesis. Under homeostatic conditions c-FMS proteolysis is initiated by the engagement of M-CSF to c-FMS. Small fragments (called FICD) are generated by c-FMS proteolysis at a later phase of c-FMS activation. FICD forms a complex with DAP5 (eIF4G2) and its activity is integrated with MNK1/2 to promote eIF4E activation. Upon stimulation with RANKL, FICD interacts with protein translation and gene expression pathway proteins to drive NFATc1 protein expression and osteoclastogenesis. Under high-MCSF conditions, such as rheumatoid arthritis synovium, constitutive c-FMS signaling augments and maintains FICD generation and thus promote osteoclast differentiation. Therefore, our findings suggest that c-FMS proteolysis may be differentially regulated under inflammatory and homeostatic conditions to fine-tune osteoclast differentiation and function.

FIG. 20 shows generation of DDK-tagged FICD. DDK-tagged FICD was generated based on N-terminal sequencing by MASS spectrophotometer analysis and a protease cleavage site prediction program. Arrow indicates the potential protease cleavage sites. FICD regions (a.a. 631-972) were then cloned into pMX-puro to generate pMX-puro-FICD.

### DETAILED DESCRIPTION OF THE INVENTION

Described herein is a novel mechanism by which rheumatoid arthritis (RA) osteoclast precursors accelerate osteoclast differentiation and bone erosion and the pathophysiological importance of these mechanisms in in vivo arthritic bone destruction. The compositions and methods described herein relate to a new protein marker, termed FMS IntraCellular Domain (FICD) fragments, that closely correlates with increased osteoclastic bone loss.

Ectodomain shedding is critical for the function of various membrane proteins. Many cell surface proteins such as Notch undergo proteolysis by regulated intracellular proteolysis (called RIP) and generate functional small fragments of membrane-anchored proteins (Kuhnle, N., Dederer, V. & Lemberg, M. K. Intramembrane proteolysis at a glance: from signalling to protein degradation. J Cell Sci 132, doi:10.1242/jcs.217745 (2019)). This process is mediated by ADAM metalloproteases and γ-secretase. c-FMS also undergoes proteolysis by TACE and γ-secretase and generates small fragments that degrade once cells are exposed to an inflammatory stimulus (Ivashkiv, L. B., Zhao, B., Park-Min, K. H. & Takami, M. Feedback inhibition of osteoclastogenesis during inflammation by IL-10, M-CSF receptor shedding, and induction of IRF8. Ann N Y Acad Sci 1237, 88-94, doi:10.1111/j.1749-6632.2011.06217.x (2011). Vahidi, A., Glenn, G. & van der Geer, P. Identification and mutagenesis of the TACE and gamma-secretase cleavage sites in the colony-stimulating factor 1 receptor. Biochemical and biophysical research communications 450, 782-787, doi:10.1016/j.bbrc.2014.06.061 (2014)). Proteolysis of c-FMS is believed to cause the breakdown and termination of its functions (Glenn, G. & van der Geer, P. CSF-1 and TPA stimulate independent pathways leading to lysosomal degradation or regulated intramembrane proteolysis of the CSF-1 receptor. FEBS Lett 581, 5377-5381, doi:10.1016/j.febslet.2007.10.031 (2007)). Due to the importance of c-FMS in myeloid cells, the functions and downstream signaling pathways of c-FMS and its interacting ligands, have been studied intensively. Despite this, the role of c-FMS proteolysis heretofore remained largely unknown.

The findings herein highlight the importance of c-FMS proteolysis in c-FMS mediated signaling pathways in OCPs/osteoclasts, and identify the mechanisms by which FICD generation and nuclear translocation occur. Also identified herein is a new pathway in which osteoclast differentiation and activity are enhanced in the pathogenesis of osteoclast-mediated bone diseases.

It is demonstrated herein that ligand engagement of c-FMS generated FMS IntraCellular Domain (FICD) fragments in both human and mouse osteoclast precursors (OCPs) by proteolysis. Increased FICD proteins in arthritic synovial macrophages promoted osteoclast differentiation and arthritic bone erosion. Using a gain-of and loss-of function study, it is demonstrated that FICDs enhanced osteoclast differentiation and activity. Furthermore, myeloid specific FICD transgenic mice exhibited an osteoporotic phenotype with increased osteoclasts and promoted arthritic bone erosion compared with control mice. This positive role of FICD in osteoclasts was mediated by accelerating MNK1/2 activation and NFATc1 expression via binding to DAP5. Overall, these findings elucidate the molecular mechanisms of c-FMS proteolysis in osteoclasts and reveal how c-FMS proteolysis accelerates the RANKL-induced osteoclast differentiation program and arthritic bone erosion. These results provide a new therapeutic target for pathological bone resorption in RA.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human. The subject may be of any age, as determined by the health care provider. In certain embodiments described herein, the patient is a subject who has or is at risk of developing a skeletal disease. The subject may have been treated for a skeletal disease previously, or is currently being treated for the skeletal disease. In one embodiment, the subject is a female. In one embodiment, the subject is a pre-menopausal woman. In another embodiment, the subject is a post-menopausal woman. In one embodiment, the subject is an older adult, e.g., over the age of 40. In another embodiment, the subject is at least 45, 50, 55, or 60 years of age. In yet another embodiment, the subject is a senior adult, i.e., over 60 years of age.

As used herein, the term “bone resorption (or loss) associated with osteoclastic activity” refers to the process by which osteoclasts break down the tissue in the bones and release the minerals into the bloodstream. Skeletal health is maintained by bone remodeling, a process in which microscopic sites of effete or damaged bone are degraded on bone surfaces by osteoclasts and subsequently replaced by new bone, which is laid down by osteoblasts. This normal process can be disturbed in a variety of pathologic processes, including localized or generalized inflammation, metabolic and endocrine disorders, primary and metastatic cancers, and during aging as a result of low-grade chronic inflammation. Abnormal bone resorption is a hallmark of many skeletal diseases.

As used herein, the term “skeletal disease” or “skeletal disorder” refers to any condition associated with the bone or joints, including those associated with bone loss, bone fragility, or softening, or aberrant skeletal growth. In some embodiments, the term “skeletal disease” refers to a condition associated with osteoclastic activity and/or bone loss. Skeletal diseases include, without limitation, osteoporosis and osteopenia, rheumatoid arthritis, osteoarthritis, psoriatic arthritis, periodontitis, periprosthetic loosening, osteomalacia, hyperparathyroidism, Paget disease of bone, spondyloarthritis, and lupus. In one embodiment, the skeletal disease is osteoporosis. In one embodiment, the skeletal disease is osteopenia. In another embodiment, the skeletal disease is rheumatoid arthritis.

“Sample” as used herein means any biological fluid or tissue that contains cells or tissue, including blood cells, fibroblasts, and skeletal muscle. In one embodiment, the sample is whole blood. In another embodiment, the sample is peripheral blood mononuclear cells (PBMC). In some embodiments, the sample contains CD14+ macrophages. In another embodiment, the sample is synovial fluid. Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means.

As used herein, the term “a therapeutically effective amount” refers an amount sufficient to achieve the intended purpose. For example, an effective amount of a therapy for bone loss associated with osteoclastic activity is sufficient to decrease osteoclastogenesis or osteoclast function, bone resorption or destruction in a subject. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

As used herein, “disease”, “disorder” and “condition” are used interchangeably, to indicate an abnormal state in a subject.

“Control” or “control level” as used herein refers to the source of the reference value for FICD or c-FMS levels. In some embodiments, the control subject is a healthy subject with no disease/bone loss. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom.

Osteoclastogenesis is the formation of bone-resorbing cells, called osteoclasts, from precursor cells of myeloid origin. A physical contact of precursor cells with osteoblasts or other specific mesenchymal cells, such as stromal or synovial cells, is essential for osteoclastogenesis. Osteoclasts are the exclusive cell type responsible for bone resorption in both bone homeostasis and pathological bone destruction. Ligand engagement of c-FMS generated FMS IntraCellular Domain (FICD) fragments in both human and mouse osteoclast precursors (OCPs) by proteolysis. It is demonstrated herein that increased FICD proteins in arthritic synovial macrophages promoted osteoclast differentiation and arthritic bone erosion. As provided herein, the presence or number of FICDs in CD14+ cells provides a marker for increased osteoclastic activity in bone. As shown in the examples, the frequency of FICDs in CD14+ cells in synovial fluid from RA patients was determined to be significantly greater than healthy or osteoarthritis patients.

Thus, in one aspect, a method of treating bone resorption associated with osteoclastic activity in a subject in need thereof is provided. The method includes reducing the level of FMS intracellular fragments (FICDs) in the subject.

In one embodiment, the method includes administering an effective amount of an inhibitor of MNK1/2 to a subject. Mitogen-activated protein kinases-interacting kinases 1 and 2 (Mnk1/2) are Ser/Thr kinases from the Ca²⁺/calmodulin-dependent kinase family. They both activate the eukaryotic initiation factor 4E (eIF4E) by phosphorylating it at the conserved Ser209. In one embodiment, the MNK inhibitor is MNK1 inhibitor. In another embodiment, the inhibitor is a MNK2 inhibitor. In another embodiment, the inhibitor is a pan-MNK inhibitor. Such MNK1/2 inhibitors are known in the art and include, without limitation, CGP-57380, timovosertib (eFT-508), ETC-206, SLV-2436, and cercosporamide. In one embodiment, the MNK inhibitor is CGP-57380. In another embodiment, the MNK inhibitor is timovosertib. In another embodiment, the MNK inhibitor is SLV-2436. In another embodiment, the MNK inhibitor is ETC-206. In another embodiment, the MNK inhibitor is cercosporamide.

As described herein, calpain 1 regulates FICD generation. Thus, in one embodiment, the method includes administering an effective amount of an inhibitor of calpain 1 or pan-Calpain inhibitor to a subject. Such inhibitors include, without limitation, BDA-410, PD 151746, ALLM, MDL-28170, calpeptin, ALLN, PD 150606, calpain inhibitor XII, Z-L-Abu-CONH-ethyl, and Z-L-Abu-CONH(CH2)3-morpholine. In one embodiment, the calpain inhibitor is BDA-410. In another embodiment, the calpain inhibitor is PD 151746. In another embodiment, the calpain inhibitor is ALLM. In another embodiment, the calpain inhibitor is MDL-28170. In another embodiment, the calpain inhibitor is ALLN. In another embodiment, the calpain inhibitor is PD 150606. In another embodiment, the calpain inhibitor is calpain inhibitor XII. In another embodiment, the calpain inhibitor is Z-L-Abu-CONH-ethyl. In another embodiment, the calpain inhibitor is Z-L-Abu-CONH(CH2)3-morpholine. In another embodiment, the calpain inhibitor is calpeptin.

When osteoclastic remodeling is present, FICDs are produced in synovial CD14+ cells from cleavage of c-FMS to soluble c-FMS by TACE. In another embodiment, the method includes inhibiting TNF-alpha converting enzyme (TACE). The TACE cleavage site of c-FMS (also called CSF-1) has been identified. Vahidi et al, Identification and mutagenesis of the TACE and c-secretase cleavage sites in the colony-stimulating factor 1 receptor, Biochemical and biophysical research communications 450, 782-787 (2014)). In one embodiment, a blocking peptide is provided which binds the TACE proteolytic domain which normally recognizes the cleavage site of c-FMS. In one embodiment, the blocking peptide has a sequence comprising ALMSEL with up to 3 amino acid substitutions. In another embodiment, the blocking peptide has a sequence comprising AHADEKEALMSELK with up to 5 amino acid substitutions. In another embodiment, the blocking peptide has a sequence comprising at least 8 consecutive residues of the sequence, AHADEKEALMSELK with up to 3 amino acid substitutions. In one embodiment, the blocking peptide has a sequence comprising GQSKQ with up to 2 amino acid substitutions. In another embodiment, the blocking peptide has a sequence comprising FRAVSLGQSQLP with up to 5 amino acid substitutions. In another embodiment, the blocking peptide has a sequence comprising at least 6 consecutive residues of the sequence, FRAVSLGQSQLP with up to 3 amino acid substitutions.

The diagnosis of bone loss and assessment of fracture risk are based on the quantitative analysis of bone mineral density (BMD) by dual-energy x-ray absorptiometry (DXA) (G. M. Blake, I. Fogelman, The role of DXA bone density scans in the diagnosis and treatment of osteoporosis. Postgrad Med J 83, 509-517 (2007), incorporated herein by reference). However, the gold standard method of BMD assessment of bone mass by DXA only partially provides information about bone strength. In addition, changes in radiographically detectable bone mass may be delayed from several months to more than a year for specific insults or treatments that affect bone mass. Therefore, a readout that responds more rapidly to changes in bone physiology is desired. Thus, in another aspect, a method of diagnosing and treating bone loss associated with osteoclastic activity in a subject is provided. In one embodiment, the method includes (i) quantifying the amount of FICDs in a sample from the subject and (ii) diagnosing a bone loss in the subject when an increase in FICDs is detected as compared to a control. In one embodiment, the method includes treating the subject for the bone loss.

The presence or level of FICDs in a sample may be determined by the person of skill in the art, e.g., by the use of ELISA. In one embodiment, the FICDs are detected using an antibody directed to the C-terminus of c-FMS. Antibodies useful in detecting the presence or level of FICDs are known in the art and include, e.g., sc-365719 (Santa Cruz), 102-17447 (RayBiotech), Cell signaling #3152, Santacruz C-20, and D-8, or may be developed. Other methods for detecting FICDs in a sample include, e.g., immunoprecipitation, immunoelectrophoresis, and spectrometry methods such as HPLC and LC/MS.

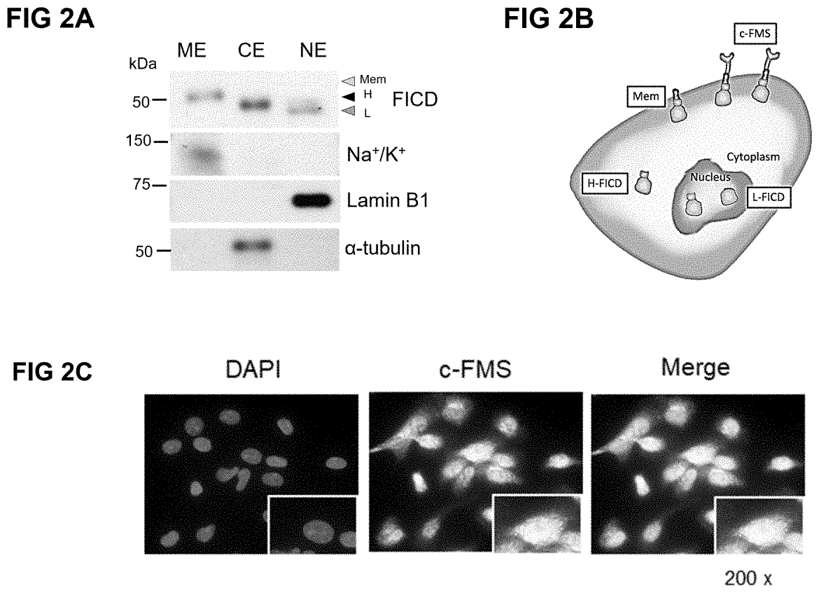

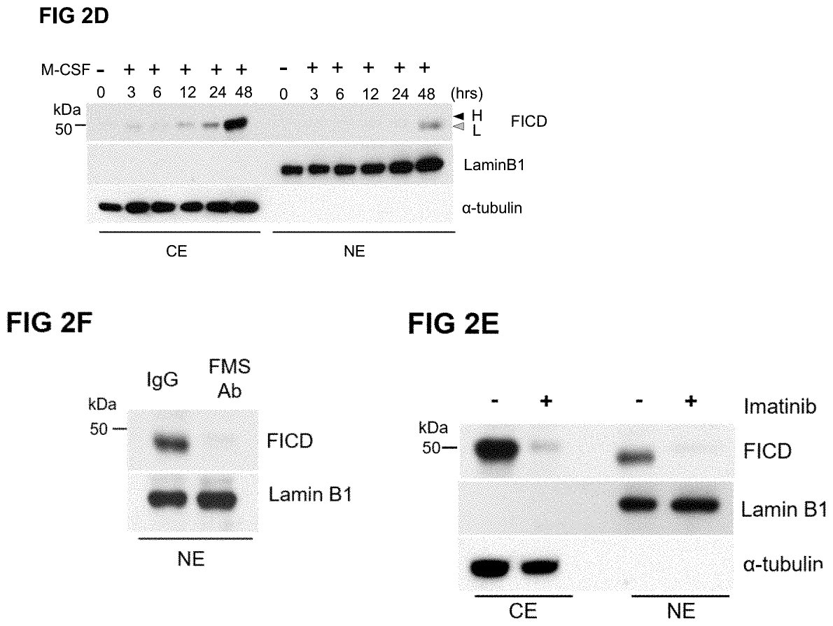

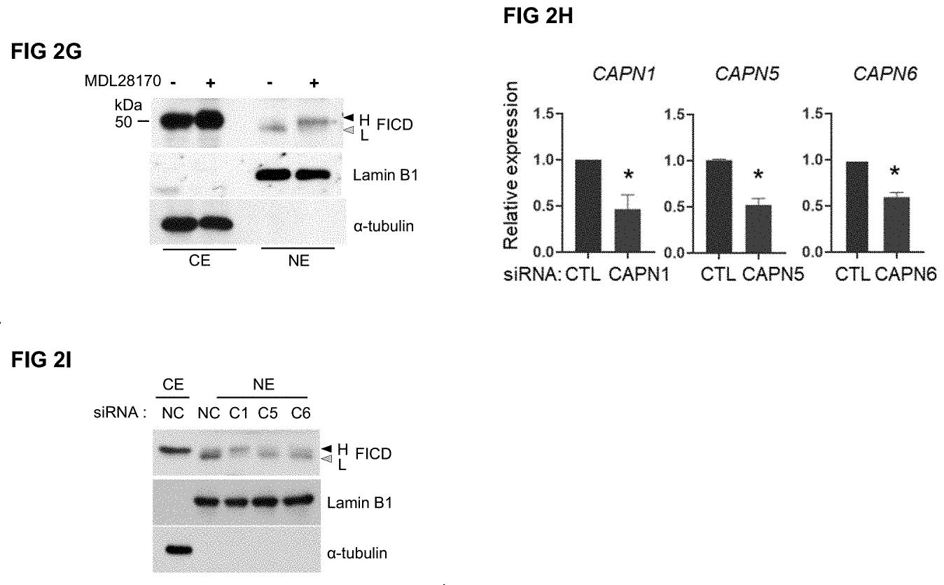

As described herein, FICD are observed in three sizes, based on their location in the cell: membrane, cytoplasm, and nucleus. FIG. 2A. The membrane-bound form has the highest molecular weight of FICD (mem), followed by the slightly smaller cytoplasmic FICD which is denoted high molecular mass FICD (H-FICD). Both forms are larger than nuclear FICD, which is denoted L-FICD for low molecular mass FICD. In one embodiment, the FICD has a molecular weight of approximately 48 kD. In another embodiment, the FICD has a molecular weight of approximately 50 kD.

In one embodiment, a sample of synovial fluid containing CD14+ cells is obtained from a subject. The CD14+ cells are isolated or concentrated using conventional methods, such as FACS. The CD14+ cells are lysed and contacted with antibodies directed to the C-terminal portion of c-FMS. The amount of FICD-bound antibodies is then calculated using routine methods. An increase in the amount of FICD in the sample, as compared to a control, is indicative of bone loss associated with osteoclastic activity.

When c-FMS is cleaved to generate FICD in cells, soluble c-FMS remains in the blood. Thus, in another embodiment, a method of diagnosing and treating bone loss associated with osteoclastic activity in a subject is provided which includes (i) quantifying the amount of circulating soluble c-FMS in a sample from the subject; and (ii) diagnosing a bone loss in the subject when an increase in soluble c-FMS is detected as compared to a control. The method includes treating the subject for the bone loss.

In one embodiment, a blood sample is obtained from a subject and contacted with antibodies directed to the central or N-terminal portion of c-FMS. The amount of soluble c-FMS-bound antibodies is then calculated using routine methods, such as ELISA as described herein. Suitable antibodies directed to the N-terminus of c-FMS are known in the art, such as R&D systems Clone #61701 or Santa Cruz H-300, or may be developed. Other methods for detecting soluble c-FMS in a sample include, e.g., immunoprecipitation, immunoelectrophoresis, and spectrometry methods such as HPLC and LC/MS.

In another aspect, a method of diagnosing a bone loss associated with osteoclastic activity in a subject is provided. The method includes one or more of: identifying the presence of FICDs in a sample from a subject and quantifying the amount of FICDs in the sample. In one embodiment, the sample is synovial fluid. In some embodiments, the presence or amount of FICDs is detected in a sample obtained from a subject. In one embodiment, the level of FICDs is compared to a control level. In one embodiment, detection of, or an increase in the number of FICDs, as compared to a control indicates the presence of a bone loss associated with osteoclastic activity. In one embodiment, the bone loss associated with osteoclastic activity is associated with osteoporosis. In another embodiment, the bone loss associated with osteoclastic activity is associated with rheumatoid arthritis. In some embodiments, the control subject is a healthy subject with no disease. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom. In one embodiment, the method further includes treating the subject for bone loss.

In another aspect, a method of diagnosing a bone loss associated with osteoclastic activity in a subject is provided. The method includes one or more of: identifying the presence of soluble c-FMS in a sample from a subject and quantifying the amount of c-FMS in the sample. In one embodiment, the sample is whole blood. In another embodiment, the sample is PBMC. In another embodiment, the sample is blood serum. In another embodiment, the sample contains CD14+-derived macrophages. In some embodiments, the presence or amount of soluble c-FMS is detected in a sample obtained from a subject. In one embodiment, the level of soluble c-FMS is compared to a control level. In one embodiment, detection of, or an increase in the number of soluble c-FMS, as compared to a control indicates the presence of a bone loss associated with osteoclastic activity. In one embodiment, the bone loss associated with osteoclastic activity is associated with osteoporosis. In another embodiment, the bone loss associated with osteoclastic activity is associated with rheumatoid arthritis. In some embodiments, the control subject is a healthy subject with no disease. In yet other embodiments, the control or reference is the same subject from an earlier time point. Selection of the particular class of controls depends upon the use to which the diagnostic/monitoring methods and compositions are to be put by the care provider. The control may be a single subject or population, or the value derived therefrom. In one embodiment, the method further includes treating the subject for bone loss.

In one embodiment, the method of diagnosing bone loss includes treatment with an appropriate therapeutic. Such therapeutics include anti-resorptive therapy, such as Bisphosphonates including Alendronate (Fosamax), Risedronate (Actonel), Ibandronate (Boniva), and Zoledronic acid (Reclast). Other useful therapeutics include disease-modifying anti-rheumatic drugs (DMARDs). DMARDs include, without limitation, ciclosporin, cyclophosphamide, hydroxychloroquine, leflunomide, methotrexate, mycophenolate, and sulfasalazine. Other therapies include, without limitation, nonsteroidal anti-inflammatory drugs (NSAIDs), steroids such as prednisone, methotrexate (Trexall, Otrexup, others), leflunomide (Arava), hydroxychloroquine (Plaquenil) and sulfasalazine (Azulfidine), abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rittman), sarilumab (Kevzara), tocilizumab (Actemra) and tofacitinib (Xeljanz). Other additional therapies include Other therapies include hormone like medications including raloxifene (Evista), Denosumab (Prolia, Xgeva), Teriparatide (Forteo), Abaloparatide (Tymlos).

In another aspect, a method of assessing the efficacy of a treatment for bone loss is provided. In one embodiment, a baseline level of FIDCs or soluble c-FMS is obtained from the subject prior to, or at the beginning of treatment for bone loss. After a desirable time period, the level of FIDCs or soluble c-FMS in the subject is measured again. A decrease in the level of FIDCs or soluble c-FMS as compared to the earlier time point indicates that the treatment for bone loss is, at least partially, efficacious. The treatment may be any of those described herein, or other treatments deemed suitable by the health care provider. In one embodiment, the treatment regimen is altered based on the level of FIDCs or soluble c-FMS detected. In one embodiment, the second time point is at least 6 months, 12 months, 18 months, 2 years, 3, years, 4 years, 5, years or more after the first time point.

In any of the methods described herein, the subject may have, or be suspected of having or developing, a skeletal disease, as described hereinabove. In one embodiment, the subject has, or is suspected of having or developing, rheumatoid arthritis. In another embodiment, the subject has, or is suspected of having or developing, psoriatic arthritis. In another embodiment, the subject has, or is suspected of having or developing, periodontitis. In another embodiment, the subject has, or is suspected of having or developing, periprosthetic loosening. In another embodiment, the subject has, or is suspected of having or developing, osteoporosis. In another embodiment, the subject has, or is suspected of having or developing, bone metastasis.

In one embodiment, a method of diagnosing and treating skeletal disease in a subject is provided. The method comprises one or more of quantifying the amount of FICDs in a sample from the subject; quantifying the number of circulating soluble c-FMS in a sample from the subject; diagnosing the skeletal disease in the subject when an increase in FICDs or soluble c-FMS is detected as compared to a control; and treating the subject for the bone loss. In one embodiment, the skeletal disease is osteoporosis or osteopenia. In another embodiment, the skeletal disease is rheumatoid arthritis. In one embodiment, the subject is treated for the skeletal disease using antiresorptive therapy.

Unless defined otherwise in this specification, technical and scientific terms used herein have the same meaning as commonly understood by one of ordinary skill in the art and by reference to published texts, which provide one skilled in the art with a general guide to many of the terms used in the present application.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

The following examples are illustrative only and are not intended to limit the present invention.

### EXAMPLES

Osteoporosis is a metabolic bone disorder that compromises bone strength and leads to an increased risk of fracture. Skeletal fractures caused by osteoporosis lead to morbidity and an increased risk of mortality; such fractures are also associated with expensive care costs. Thus, osteoporosis represents a serious public health problem, and both early diagnosis and effective therapies for osteoporosis are urgently needed. However, current diagnostic methods are not suitable to detect the risk of fracture early, and the available anti-resorptive drugs that are effective in inhibiting bone resorption have significant side effects. As described herein, the inventors have developed early diagnostic biomarkers of osteoporosis or pathological bone loss.

The key findings are as follows:

1. c-FMS generated an essential signal for the differentiation and function of macrophages/osteoclasts, and the importance of c-FMS/M-CSF signaling has been implicated in multiple aspects of macrophage/osteoclast biology. We discovered that M-CSF triggered the activation of c-FMS proteolysis and generated small cleaved fragments (called FMS IntraCellular Domain (FICD)). Consistent with previous reports, we also found increased levels of M-CSF in RA synovial fluids. Overall, our results link high levels of M-CSF in RA synovium to high FICD expression in RA OCPs.

2. Using pharmacological and genetic approaches, we established the pathophysiological importance of FICD and associated pathways. Increased FICD was found in synovial CD14+ cells from patients with RA. To model this in vivo, we generated conditional FICD knock-in mice. Conditional expression of FICDs in myeloid lineage cells resulted in significantly increased osteoclastogenesis and bone erosion in an arthritis model. Our data suggest that increased FICDs may contribute to arthritic bone erosion in patients with RA.

3. Our study, for the first time, identified c-FMS proteolysis as a positive regulator of osteoclastogenesis. c-FMS proteolysis is regulated by c-FMS conventional signals; c-FMS proteolysis was induced by M-CSF/c-FMS engagement and was blocked when c-FMS signaling was inhibited. Our findings provide a novel component of the c-FMS-mediated signaling cascade and a comprehensive overview of the role of c-FMS in macrophages and osteoclasts.

4. Our study provides both in vivo and in vitro data to support a novel signaling pathway mediated by FICDs. FICDs formed a complex with DAP5 and further activated eIF4E phosphorylation, which we linked to increased expression of NFATc1, a master regulator of osteoclastogenesis. We also showed that modulating each component including DAP5 or MNK1/2 activation suppressed osteoclastogenesis and affected arthritic bone erosion in a K/BXN serum transfer arthritis model. The role of the FICD/DAP5/MNK1/2/eIF4E axis in osteoclasts was previously unknown. Our study addresses the importance of this new pathway in osteoclasts. Furthermore, to the best of our knowledge, this is the first study to show the efficacy of MNK1/2 inhibition on arthritic bone erosion.

Osteoclasts are bone-resorbing cells derived from the myeloid lineage cells that are responsible for arthritic bone erosion (Tsukasaki, M. & Takayanagi, H. Osteoimmunology: evolving concepts in bone-immune interactions in health and disease. Nat Rev Immunol 19, 626-642, doi:10.1038/s41577-019-0178-8 (2019). Park-Min, K. H. Mechanisms involved in normal and pathological osteoclastogenesis. Cell Mol Life Sci 75, 2519-2528, doi:10.1007/s00018-018-2817-9 (2018). Novack, D. V. & Teitelbaum, S. L. The osteoclast: friend or foe? Annu Rev Pathol 3, 457-484, doi:10.1146/annurev.pathmechdis.3.121806.151431 (2008)). There are many cellular sensor and effector proteins that play a role in the generation and ultimate function of osteoclasts. Of those, M-CSF and receptor activator of NF-κB ligand (RANKL) are essential factors for the function and differentiation of monocytes and osteoclasts (Park-Min, K. H. Mechanisms involved in normal and pathological osteoclastogenesis. Cell Mol Life Sci 75, 2519-2528, doi:10.1007/s00018-018-2817-9 (2018). Novack, D. V. & Teitelbaum, S. L. The osteoclast: friend or foe? Annu Rev Pathol 3, 457-484, doi:10.1146/annurev.pathmechdis.3.121806.151431 (2008). Hamilton, J. A. Colony-stimulating factors in inflammation and autoimmunity. Nat Rev Immunol 8, 533-544, doi:10.1038/nri2356 (2008). Ross, F. P. & Teitelbaum, S. L. alphavbeta3 and macrophage colony-stimulating factor: partners in osteoclast biology. Immunol Rev 208, 88-105, doi:10.1111/j.0105-2896.2005.00331.x (2005)). M-CSF signaling induces expression of the receptor activator of NF-κB (RANK), a receptor for RANKL, and RANKL induces the expression of NFATc1, a master regulator of osteoclastogenesis, to initiate the osteoclast differentiation program (Tsukasaki, M. & Takayanagi, H. Osteoimmunology: evolving concepts in bone-immune interactions in health and disease. Nat Rev Immunol 19, 626-642, doi:10.1038/s41577-019-0178-8 (2019)).

Transcriptional factor networks involved in NFATc1 mRNA expression are well-characterized, but the regulatory mechanisms of NFATc1 protein expression remain unclear. mRNA translation is tightly controlled at multiple levels, and altered protein synthesis can lead to disease or cell apoptosis (Gebauer, F. & Hentze, M. W. Molecular mechanisms of translational control. Nat Rev Mol Cell Biol 5, 827-835, doi:10.1038/nrm1488 (2004).). The initiation of protein synthesis is a rate-limiting step. This is facilitated by eIF4F, which binds to the 5′cap, m7GTP, of mRNAs, recruiting mRNA to the ribosome. eIF4F is a multi-subunit protein complex, composed of eIF4A, eIF4E, and eIF4G. eIF4G recruits to mRNA, the 43S preinitiation complex consisting of three protein family members: eIF4GI (eIF4G1), eIF4GII, and DAP5 (eIF4G2). In contrast to the well-known function of eIF4G1, the role of DAP5 in protein translation is controversial. A recent study showed that DAP5 can form inactive complexes and suppress protein translation (Imataka, H., Olsen, H. S. & Sonenberg, N. A new translational regulator with homology to eukaryotic translation initiation factor 4G. The EMBO journal 16, 817-825, doi:10.1093/emboj/16.4.817 (1997).). Another study revealed that the DAP5 complex promotes alternative translation of specific subsets of mRNA (Yoffe, Y. et al. Cap-independent translation by DAP5 controls cell fate decisions in human embryonic stem cells. Genes Dev 30, 1991-2004, doi:10.1101/gad.285239.116 (2016)). Beyond this, the full function of DAP5 complex has not been defined and the role of DAP5 in myeloid cells is unknown.

### Example 1: Materials and Methods

### Mice

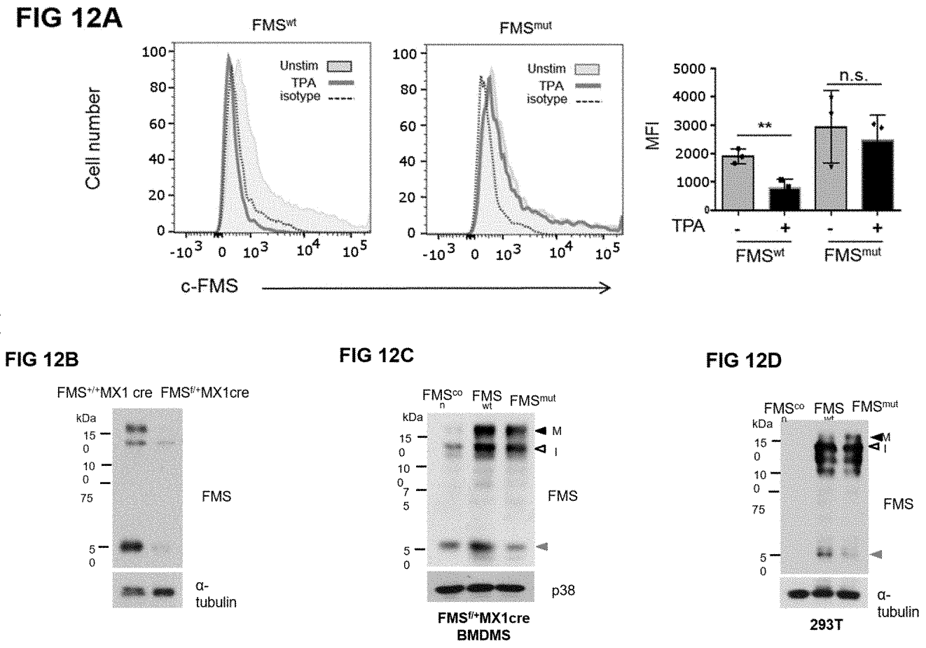

Human c-FMS fragment (FICD) knock-in mice (FICD^{KI/KI}) were generated and purchased from Cyagen Biosciences Inc. (Guangzhou, Guanddong, China). Briefly, mouse genomic fragments containing homology arms of ROSA26 allele were amplified from BAC clone using PCR and Neo (positive selection marker) flaked by SDA (self-deletion anchor) and CAG-loxP-3*polyA-loxP, and human CSF1R intracellular domain-poly A cassette (NM_005211.3) were assembled into targeting vector shown in FIG. 12A. Targeting vector (pRP.ExBiEF1A-loxp-stop-loxp-hFICD) were then linearized by Not I digestion and electrophorated into C57BL/6J ES cells. Six positive G418 resistant ES clones were selected and further confirmed by Southern blot (FIG. 12B). The G418-resistant ES clones were then transfected with FLP (flippase) to remove the Neo drug marker. Targeted ES cells were injected into mouse blastocysts and were transferred into surrogate mothers. Male chimera was bred with C57BL/6J female to generate F1 heterozygous mice. F1 mice were crossed to each other to generate wild type, heterozygous, and homozygous (FICD^{KI/KI}) mice. FICD^{KI/KI} mice were crossed to Lys2-Cre mice (The Jackson Laboratory) to generate FICD^{KI/KI} LysM-Cre (LysM^{cre/+}×FICD^{KI/KI}) mice. Age-and gender matched littermate LysM^{cre/+}FICD^{+/+} mice were used as controls. 8 week-old female LysM^{cre/+}×FICD^{KI/KI} mice were randomly assigned for K/BXN serum transfer model, while 12 week-old male LysM^{cre/+}×FICD^{KI/KI} mice were used for micro-CT analysis. C57BL/6J female mice were obtained from Jackson Laboratory and were randomly allocated for in vitro experiments. Both Csf1r^{fl/fl} mice and Mx1-Cre transgenic mice were purchased from The Jackson Laboratory. Csf1r^{fl/fl} mice were crossed to Mx1-Cre transgenic mice to generate Mx1 Cre Csf1r^{fl/+} mice to diminish intracellular FICD generation. c-FMS^{flox/+} Mx1cre(+) mice (referred to as c-FMS^{hetΔMX} mice) and littermate control c-FMS^{WT/WT} Mx1cre(+) mice were used for the experiments. To induce FMS deletion, 300 mg of Poly (I:C) (Thermo Fisher Scientific) was injected three times at age of 6 weeks. LysM Cre mice were crossed with Raptor^{fl/fl} mice to generate either LysM^{Cre/Cre} Raptor^{+/+} or LysM^{Cre/Cre} Raptor^{+/+} mice. All animals were randomly assigned into experimental groups. Animals were housed in a specific pathogen-free environment in the Weill Cornell Medicine vivarium and all the experiments conformed to the ethical principles and guidelines approved by the Institutional and Animal Care and Use Committee of Weill Cornell Medical College.

### Human Studies

Human synovial fluid (SF) samples were collected from RA and osteoarthritis (OA) patients as previously described (Gordon R A, Grigoriev G, Lee A, Kalliolias G D, Ivashkiv L B. The interferon signature and STAT1 expression in rheumatoid arthritis synovial fluid macrophages are induced by tumor necrosis factor alpha and counter-regulated by the synovial fluid microenvironment. Arthritis and rheumatism 64, 3119-3128 (2012)). Patients SF from active effusions was obtained from 24 patients with RA, and 10 patients with OA. The protocol was approved by the Hospital for Special Surgery Institutional Review Board (2016-957, 2016-958, and 2016-139). Active effusion was defined as an acute noninfectious inflammatory SF accumulation attributed to a flare of RA that required arthrocentesis based on medical indications. The diagnosis of RA was based on the 1987 revised criteria of the American College of Rheumatology (Arnett F C, et al. The American Rheumatism Association 1987 revised criteria for the classification of rheumatoid arthritis. Arthritis and rheumatism 31, 315-324 (1988)). There was limited information about patients' medications, and correlation of our findings with therapy was not possible.

### Cells

Peripheral blood mononuclear cells (PBMCs) from blood leukocyte preparations purchased from the New York Blood Center or mononuclear cells from SF of RA patients were isolated by density gradient centrifugation with Ficoll (Invitrogen, Carlsbad, Calif.). CD14⁺ cells were obtained by isolation using anti-CD14 magnetic beads, as recommended by the manufacturer (Miltenyi Biotec, CA). Human CD14⁺ cells were cultured in α-MEM medium (Invitrogen) supplemented with 10% fetal bovine serum (FBS, Hyclone; SH30070.03) and 1% L-glutamin with 20 ng/ml of M-CSF for 12 hours to generate osteoclast precursor cells (OCPs) The purity of monocytes was >97%, as verified by flow cytometric analysis (Park-Min K H, et al. Inhibition of osteoclastogenesis and inflammatory bone resorption by targeting BET proteins and epigenetic regulation. Nature communications 5, 5418 (2014)).

For human osteoclastogenesis assays, cells were added to 96 well plates in triplicate at a seeding density of 5×10⁴ cells per well. Osteoclast precursors were incubated with 20 ng/ml of M-CSF and 40 ng/ml of human soluble RANKL up to 5 days in α-MEM supplemented with 10% FBS and 1% L-glutamine. Cytokines were replenished every 3 days. On each day, cells were fixed and stained for TRAP using the Acid Phosphatase Leukocyte diagnostic kit (Sigma; 387A) as recommended by the manufacturer. Multinucleated (greater than 3 nuclei), TRAP-positive osteoclasts were counted in triplicate wells. For mouse osteoclastogenesis, bone marrow (BM) cells were flushed from femurs of mice and cultured with murine M-CSF (20 ng/ml) on petri dishes in α-MEM supplemented with 10% FBS, 1% anti-biotics and 1% L-glutamin after lysis of RBCs using ACK lysis buffer (Gibco). Then, the non-adherent cell population was recovered the next day and cultured with M-CSF-containing conditional medium (CM) for three additional days. We defined this cell population as mouse BMDMs. Then, we plated 2×10⁴ cells per well in triplicate wells on a 96 well plate and added M-CSF (20 ng/ml) and RANKL (50 ng/ml) up to 4 days, with exchange of fresh media every 3 days. All cell-cultures were performed by a modification of the previously published method (Park-Min K H, et al. Inhibition of osteoclastogenesis and inflammatory bone resorption by targeting BET proteins and epigenetic regulation. Nature communications 5, 5418 (2014)).

### RNA Preparation and Real-Time PCR

DNA-free RNA was obtained using the RNeasy Mini Kit from QIAGEN with DNase treatment, and 0.5 μg of total RNA was reverse transcribed using a First Strand cDNA Synthesis kit (Fermentas, Hanover, Md.). Real time PCR was performed in triplicate using the iCycler iQ thermal cycler and detection system (Applied Biosystems, Carlsbad, Calif.) following the manufacturer's protocols. The primer sequences are listed in Table 1.

Real-time PCR primers

| | | SEQ |
|---|---|---|
| Gene | | ID |
| Symbol | Sequence | NO |
| hTACE | Forward: 5′-ACCCTTTCCTGCGCCCCAGA-3′ | 1 |
| | Reverse: 5′-GTTTTGGAGCTGCTGGCGCC-3′ | 2 |
| hCAPN1 | Forward: 5′-TGCCGTTTGCTGAGTGTCC-3′ | 3 |
| | Reverse: 5′-TCTCCTCCGACATCCTCGGG-3′ | 4 |
| hCAPN5 | Forward: 5′-CTCGGCCGGTGTTCCC-3′ | 5 |
| | Reverse: 5′-CCGGCGTGCCCTTATAGTAG-3′ | 6 |
| hCAPN6 | Forward: 5′-GCTGTTCCATTGAGTCTCCCA-3′ | 7 |
| | Reverse: 5′-GGGTTTCTCAGGCGAACCAT-3′ | 8 |
| hNFATc1 | Forward: | 9 |
| | 5′-CTTCTTCCAGTATTCCACCTAT-3′ | |
| | Reverse: | 10 |
| | 5′-TTGCCCTAATTACCTGTTGAAG-3′ | |
| hHPRT | Forward: 5′-GACCAGTCAACAGGGGACAT-3′ | 11 |
| | Reverse: 5′-CCTGACCAAGGAAAGCAAAG-3′ | 12 |
| hFICD | Forward: 5′-TGTCTACACGGTTCAGAGCG-3′ | 13 |
| | Reverse: 5′-GGGTAGGGATTCAGCCCAAG-3′ | 14 |
| mNfatc1 | Forward: 5′-CCCGTCACATTCTGGTCCAT-3′ | 15 |
| | Reverse: 5′-TCTCCTCCGACATCCTCGGG-3′ | 16 |
| mHprt | Forward: 5′-TCCTCAGACCGCTTTTTGCC-3′ | 17 |
| | Reverse: 5′-CTAATCACGACGCTGGGACT-3′ | 18 |
| mTnf-α | Forward: 5′-GTCAGGTTGCCTCTGTCTCA-3′ | 19 |
| | Reverse: 5′-TCAGGGAAGAGTCTGGAAAG-3′ | 20 |
| mIl-6 | Forward: | 21 |
| | 5′-AAGCCAGAGTCCTTCAGAGAGA-3′ | |
| | Reverse: | 22 |
| | 5′-GGAAATTGGGGTAGGAAGGA-3′ | |

Genotype primers

| | | SEQ |
|---|---|---|
| Gene | | ID |
| Symbol | Sequence | NO |
| hFICD- | Forward: 5′-GGTGCTTGCCTTTATGCCTTTA- | 23 |
| Region_1 | 3′ | |
| | Reverse: 5′-TGGCTGCCATGAACAAAGGTT- | 24 |
| | 3′ | |
| hFICD- | Forward: 5′- | 25 |
| Region_2 | CAGGTCGCCATAGCAACAGTACTC-3′ | |
| | Reverse: 5′- | 26 |
| | AGTCGCAGATCTGCAAGCTAATTCC-3′ | |
| hFICD- | Forward: 5′- | 27 |
| Region_3 | GGGCCATTTACCGTAAGTTATGTAACG-3′ | |
| | Reverse: 5′- | 28 |
| | GCCATTTAAGCCATGGGAAGTTAG-3′ | |
| | Forward-1: 5′- | 29 |
| | TGGACAGAGGAGCCATAACTGCAG-3′ | |
| hFICD- | Forward: 5′- | 30 |
| Region_4 | GGTACAGGCTCCCAGAAGGTTGAC-3′ | |
| | Reverse: 5′- | 31 |
| | CAACGTGCTGGTTATTGTGCTGTCT-3′ | |

Oligonucleotides

| Gene | |
|---|---|
| Symbol | Sequence |
| siRNA | Dharmacon, Cat#; D-001810-10 |
| Negative | |
| control | |
| siRNA: | Invitrogen, Cat#; HSS186181 |
| hTACE | |
| siRNA: | Dharmacon, Cat#; L005799-00-0005 |
| hCAPN1 | |
| siRNA: | Dharmacon, Cat#; L-009423-00-0005 |
| hCAPN5 | |
| siRNA: | Dharmacon, Cat#; L-009423-00-0005 |
| hCAPN6 | |
| siRNA: | Dharmacon, Cat#; L-011263-00-0005 |
| hEIF4G2 | |
| siRNA: | Dharmacon, Cat#; L-012011-00-0005 |
| hFXR1 | |
| siRNA: | Dharmacon, Cat#; L-064521-00-0005 |
| mEif4g2 | |
| siRNA: | Dharmacon, Cat#; L-045530-00-0005 |
| mFxr1 | |

### Enzyme-Linked Immunosorbent Assay (ELISA)

c-FMS and M-CSF in synovial fluids and the supernatants of cell culture was measured using sandwich ELISA kit (R&D Systems; DY329 and DY216) according to manufacturer's instructions. C-telopeptide of type I collagen (CTX-1) and Procollagen 1 N-Terminal Propeptide (P1NP) in Serum from WT and FICDtg^{M} mice were measured using RatLapstm EIA kit (Immunodiagnostic Systems; AC-06F1) or P1NP-ELISA kit (Cloud-Clone corp; SEA957Mu) according to the manufacturer's instructions.

### Measurement of Cytokine Production

IL-6 and TNF-α, in culture supernatants were assessed quantitatively by Luminex multiplex cytokine assay (R&D Systems) as described by the manufacturer.

### RNA Interference

0.2 nmol of three short interfering RNAs (siRNAs), specifically targeting human TACE (Invitrogen; HSS186181), CAPAN 1 (Dharmacon; L005799-00-0005), CAPAN 5 (Dharmacon: L-009423-00-0005), CAPAN 6 (Dharmacon: L-009423-00-0005) or control siRNA (D-001810-10) were transfected into primary human CD14⁺ monocytes with the Amaxa Nucleofector device set to program Y-001 using the Human Monocyte Nucleofector kit (Amaxa), as previously described (Park-Min K H, et al. FcgammaRIII-dependent inhibition of interferon-gamma responses mediates suppressive effects of intravenous immune globulin. Immunity 26, 67-78 (2007)).

Bone marrow derived macrophages (BMDMs) from wild type mice were plated 2×10⁵ /ml (24 well plate) and were cultured with 20 ng/ml M-CSF for 24 hours. Cells were transfected with the 100 nM siRNA mouse Control DAP5 (Dharmacon: L-064521-00-0005) or Fxr1(Dharmacon: L-045530-00-0005) with TranslT-TKO® Transfection Reagent/opti-men. After 4 hours, add 500 ul Opti-MEM with M-CSF (20 ng/ml) and FBS (final con. 5%) serum 48 hours. and then changed complete medium with 10 ng/ml M-CSF and 50 ng/ml RANKL for 24 hours. Cells were lysed.

### Immunoblot

Whole cell extracts were prepared by lysis in buffer containing 1× Lamin sample buffer (Bio-rad) and 2-Mercaptoethanol (Sigma) or RIPA buffer (Sigma). The cell membrane-permeable protease inhibitor, Pefablock (1 mM), was added immediately prior to harvest cells. The membrane proteins were extracted with Mem-PER™ Plus Membrane Protein Extraction Kit (Thermorfisher scientific; 89842) according to manufacturer's instructions. To extracts of nucleus protein, cells were incubated in buffer A (10 mM Hepes, pH 7.9, 1.5 mM MgCl₂, 10 mM KCl, 0.1 mM EDTA, 0.1 mM EGTA, proteinase inhibitor cocktail (Complete, Roche) and 1 mM DTT) for 15 min at 4° C. NP-40 was added to a final concentration of 0.5%. Nucleus was collected by centrifugation (5000 rpm, 5 min). The pellets were lysed by Bioruptor-ultrasonicator (UCD400, Diagenode) in buffer B (20 mM Hepes, pH 7.9, 0.4M NaCl, 10 mM KCl, 1 mM EDTA, 1 mM EGTA, 10% Glycerol, proteinase inhibitor cocktail and 1 mM DTT) and collected the supernatant by centrifugation (12,000 rpm, 10 min). The protein concentration of nuclear extracts was quantitated using the Bradford assay (Bio-Rad; 5000001). For immunoblot, proteins were separated on 7.5 or 10% SDS-PAGE gels, transferred to polyvinylidene difluoride membranes (PVDF, Millipore; ISEQ00010), and detected by antibodies as listed in the figure legends.

### Lentiviral and Retroviral transduction

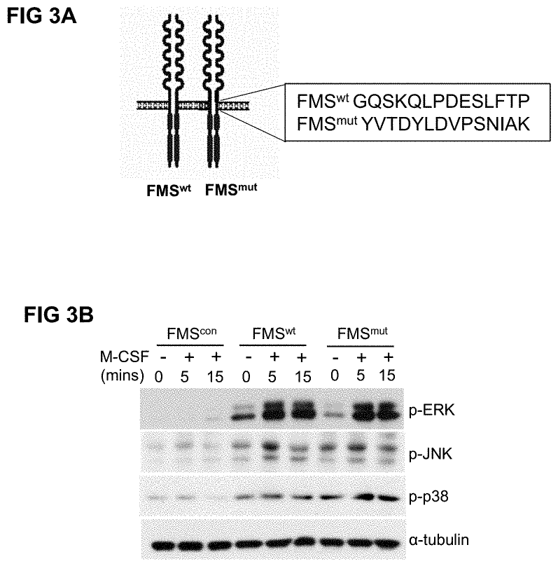

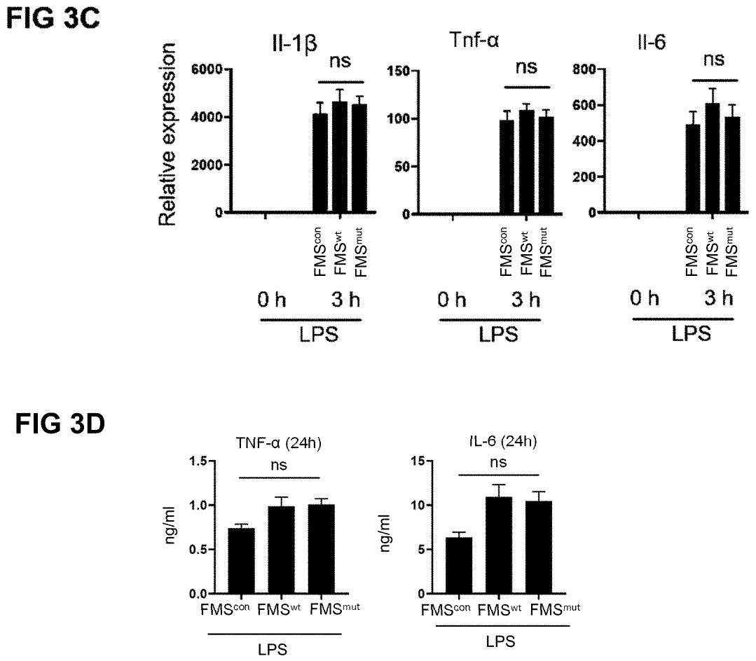

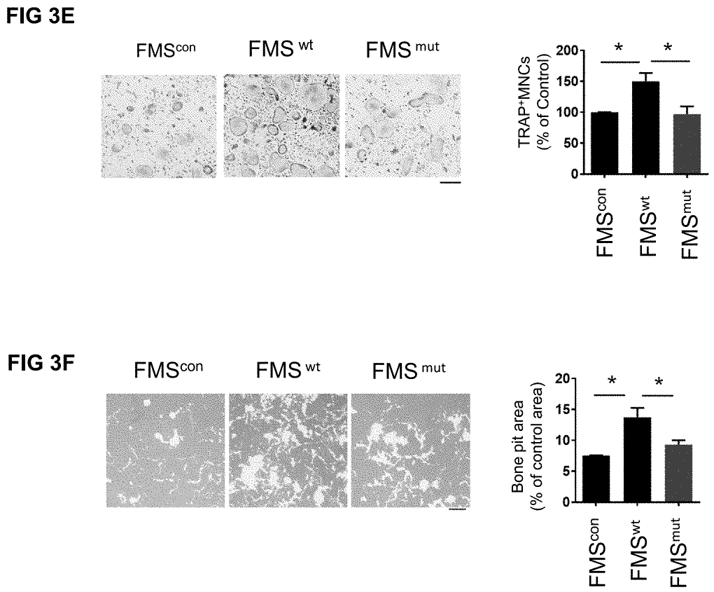

The vector containing full-length mouse c-FMS (MR211364, FMSW^{wt}) or TACE cleavage resistant c-FMS (FMS^{mut}) were purchased from Blue Heron Biotech, LLC (Origene, Md.). Briefly, FMS^{mut} generated by switching 14 amino acids from TACE cleavage sites of c-FMS with sequences from insulin receptor extracellular regions (Vahidi A, Glenn G, van der Geer P. Identification and mutagenesis of the TACE and gamma-secretase cleavage sites in the colony-stimulating factor 1 receptor. Biochemical and biophysical research communications 450, 782-787 (2014)). The target sequences were shown in FIG. 3C. FMS^{mut} and FMS^{wt} were cloned into pLenti-EF1a-C-Myc-DDK-IRES-Puro vector. 293T cells were transfected with pUC-MDG, pCMV8.9, and an empty vector (Ohno H, et al. A contact investigation of the transmission of Mycobacterium tuberculosis from a nurse working in a newborn nursery and maternity ward. Journal of infection and chemotherapy: official journal of the Japan Society of Chemotherapy 14, 66-71 (2008)), FMS^{wt}, or FMS^{mut} by Lipofectamin3000 regent (Invitrogen) to generate lentiviral particles. Supernatants were collected and concentrated with Lenti-X™ Concentrator (TaKaRa Clontec.). BMDMs from Csf1r^{f/+} Mx1-Cre⁺ male mice were cultured for 2 days with M-CSF, and then cells were transduced with lentiviral particles with 8 μg/mL polybrene (Santacruz; sc-134220). After 24 h, infected cells were re-plated for osteoclastogenesis experiment. FICD gene was amplified using PCR primers from the human c-FMS cDNA with the following primers: FICD-C; forward: 5′-GGGTCTAGAATGTCCGAGCTGAAGATC-3′ (SEQ ID NO: 32) and reverse: 5′-GGGATACCGACTGCATTAAT GCTGTT-3′ (SEQ ID NO: 33). For retrovirus transduction, FICD genes were ligated into the retroviral vector, pMX-puro (Cell Biolabs) to generate pMX-puro-FICD. The retroviral vector pMXs-puro-FICD and its control vector were transfected into a packaging cell line, Plat-E, using FuGENE HD Transfection Reagent (Promega), and then the viral supernatant was collected after 24 hours of incubation. The filtered virus-containing supernatant was mixed 6 μg/mL polybrene (Santacruz) along with 10% of M-CSF-containing conditional medium, and then added to cells. After 48 hours of viral incubation, cells were re-plated for experiments (Bae S, et al. MYC-dependent oxidative metabolism regulates osteoclastogenesis via nuclear receptor ERRalpha. The Journal of clinical investigation 127, 2555-2568 (2017)).

### Flow Cytometry

Lentiviral vector encoding control, FMS^{wt} and FMS^{mut} were used for transducing 293T cells. Cells were stimulated by 12-Otetradecanoylphorbol-13-acetate (TPA, 100 ng/ml) for 30 mins and were stained with isotype-PE (mouse IgG2a) or anti c-FMS-PE antibodies. Stained each cells were performed with a FACS Canto (BD Biosciences) and analyzed with FlowJo software (Tree Star Inc.).

### Bone-Resorption Pit Assays

Bone-resorption activity of osteoclasts was examined using 96-well Corning Osteo Assay Surface plates (Sigma). Mouse OCPs were plated at a seeding density of 1×10⁴ per well and incubated with M-CSF (20 ng/ml) and RANKL (50 ng/ml) for 5 days, with exchange of fresh M-CSF and RANKL every two days. After removing cells with 10% bleach solution, plates were stained with 1% toluidine blue solution to visualize the formation of pits. Resorbed area was analyzed using OsteoMeasure software (OsteoMetrics, Inc.).

### MASS Spectrophotometer Assay

293T cells were transfected with pCMV6-Entry-c-FMS-MYC-DDK (NM_005211, Origene, Rockville, Md.) using Lipofectamin 3000 (Thermofisher scientific). Transfected cells were incubated with M-CSF (20 ng/ml) for one day and nuclear proteins were immunoprecipitated (IP) with antibodies against N-terminal of c-FMS (Santa Cruz; H-300 and R&D systems; clone #61780) to remove full-length c-FMS as a negative selection. Subsequently, the IP-proteins were incubated with either mouse IgG or DDK-tag Ab conjugated magnetic bead (Origene). Proteins bound to ab-beads were eluted with water. Samples were subjected to SDS PAGE gel was submitted for the mass spectrophotometery assay. Mass Spectrometry assay (n=2) was performed by The Taplin Biological Mass Spectrometry Facility in Harvard Medical School. Briefly, excised gel bands were cut into approximately 1 mm³ pieces. Gel pieces were then subjected to a modified in-gel trypsin digestion procedure (Shevchenko A, Wilm M, Vorm O, Mann M. Mass spectrometric sequencing of proteins silver-stained polyacrylamide gels. Anal Chem 68, 850-858 (1996)). Gel pieces were washed and dehydrated with acetonitrile for 10 min. followed by removal of acetonitrile. Pieces were then completely dried in a speed-vac. Rehydration of the gel pieces was with 50 mM ammonium bicarbonate solution containing 12.5 ng/μl modified sequencing-grade trypsin (Promega, Madison, Wis.) at 4° C. After 45 min., the excess trypsin solution was removed and replaced with 50 mM ammonium bicarbonate solution to just cover the gel pieces. Samples were then placed in a 37° C. room overnight. Peptides were later extracted by removing the ammonium bicarbonate solution, followed by one wash with a solution containing 50% acetonitrile and 1% formic acid. The extracts were then dried in a speed-vac (˜1 hr). The samples were reconstituted in 5-10 μl of HPLC solvent A (2.5% acetonitrile, 0.1% formic acid). A nano-scale reverse-phase HPLC capillary column was created by packing 2.6 μm C18 spherical silica beads into a fused silica capillary (100 μm inner diameter×˜30 cm length) with a flame-drawn tip (Peng J, Gygi S P. Proteomics: the move to mixtures. J Mass Spectrom 36, 1083-1091 (2001)). After equilibrating the column each sample was loaded via a Famos auto sampler (LC Packings, San Francisco Calif.) onto the column. A gradient was formed and peptides were eluted with increasing concentrations of solvent B (97.5% acetonitrile, 0.1% formic acid). As peptides eluted they were subjected to electrospray ionization and then entered into an LTQ Orbitrap Velos Pro ion-trap mass spectrometer (Thermo Fisher Scientific, Waltham, Mass.). Peptides were detected, isolated, and fragmented to produce a tandem mass spectrum of specific fragment ions for each peptide. Peptide sequences (and hence protein identity) were determined by matching protein databases with the acquired fragmentation pattern by the software program, Sequest (Thermo Fisher Scientific, Waltham, Mass.) (Eng J K, McCormack A L, Yates J R. An approach to correlate tandem mass spectral data of peptides with amino acid sequences in a protein database. J Am Soc Mass Spectrom 5, 976-989 (1994)). All databases include a reversed version of all the sequences and the data was filtered to between a one and two percent peptide false discovery rate.

### The Ingenuity Pathway Analysis (IPA)

IPA was used to analyze the functions of FICD-interacting proteins obtained from mass spectrophotometry. The molecular and cellular function was used to predict the functions whose change in enrichment relative to control could explain the interaction with FICDs.

### Immunocytochemistry

Human CD14⁺-monocytes were cultured with M-CSF (20 ng/ml) in culture slide (BD Falcon; REF 354104) for 2 days. Cells were fixed with 3.7% formalin in PBS for 20 min at room temperature. Cells were permeabilized with 1% triton X-100 for 5 min, washed 3 times before blocking with solution that contains 5% horse serum, 5% Goat and 1% BSA (without IgG) in PBS for 1 h. Cells were incubated with primary antibody C-terminus specific c-FMS Ab (SantaCruz Biotechnology; sc-692) overnight at 4° C. followed by incubation with anti-rabbit Alexa Fluor 488-conjugated secondary antibody (A11008, Thermo Fisher Scientific) for 40 min in room temperature. After washed, finally cells were mounted with ProLongTMGold antifade regent with-DAPI (P36931, Invitrogen). The stained cells were imaged using a Zeiss Axioplan microscope (Zeiss) with an attached Leica DC 200 digital camera (Leica) or a confocal microscope system (Zeiss LSM 880, Laser excitation/emission: 405/425 and 488/525). To determine c-FMS in the nucleus, confocal three-dimensional Z-stacks were acquired for each sample using a a Plan-Apochromat 63×/1.4 oil Dic M27 objective (Zeiss, Germany) with a slice of increment of 0.5 μm. The images were processed with Image j-Fiji software.

### Immunoprecipitation

2×10⁶ BMDMs from LysM^{cre/cre} mice were seeded into 100 mm dish and were incubated with M-CSF (10 ng/ml) for overnight. Cells were treated with 50 ng/ml RANKL for one additional day and lysed with RIPA buffer with proteinase inhibitor cocktail. An equal amount of cell lysates were incubated with magnetic beads conjugated with anti HA-Tag antibody (Thermo Fisher scientific; 88836) for 24 h at 4° C. The beads were washed 5 times with washing buffer (20 mM HEPES [pH 7.5], 150 mM NaCl, 0.1% NP-40, 1% glycerol, protease and phosphatase inhibitors). Proteins eluted from the bead with elution buffer (pH 2.8, Prod#1858606). The sample were incubated in 95° C. for 10 mins and then were analyzed by immunoblotting.

### Micro-CT and Histomorphometry Analysis

μ-CT analysis (Bouxsein M L, Boyd S K, Christiansen B A, Guldberg R E, Jepsen K J, Muller R. Guidelines for assessment of bone microstructure in rodents using micro-computed tomography. J Bone Miner Res 25, 1468-1486 (2010)) was performed as described previously (Shim J H, et al. Schnurri-3 regulates ERK downstream of WNT signaling in osteoblasts. J Clin Invest 123, 4010-4022 (2013)), and all samples were included in the analysis conducted in a blinded manner. For μCT analysis, Prior to decalcification, femurs with intact joints were scanned by microCT, with an isotropic voxel resolution of 6 μm (μCT35, Scanco, Bruttisellen, Switzerland; 55 kVp, 145 μA, 600 ms integration time) to evaluate morphological changes in bone. Bone morphology in the femur was examined in two regions: the diaphysis and the metaphysis. For cortical bone, the volume of interest (VOI) encompassed cortical bone within a 231-slice section in the diaphysis. For trabecular bone, the VOI encompassed a 200-slice section in the metaphysis, proximal to the growth plate. To ensure exclusion of primary spongiosa in the growth plate, VOIs began 50 slices proximal to the median of the growth plate. Outcome parameters for cortical bone included thickness and tissue mineral density (TMD). Trabecular bone parameters included bone volume fraction (BV/TV), trabecular thickness (Tb.Th), trabecular separation (Tb.Sp), and trabecular TMD. 3D reconstructions were generated by stacking thresholded 2D images from the contoured region.

Histomorphometry experiment was performed with tarsal bone of vehicle or MDL28170 treated mice. Bone histomorphometric analysis was performed in a blinded, nonbiased manner using a computerized semi-automated system (Osteomeasure, TN) with light microscopy. The tarsal bones were fixed in 4% paraformaldehyde for 2 days, were decalcified with 10% neutral buffered EDTA (Sigma-Aldrich), and were embedded in a paraffin. The quantification of osteoclast was performed in paraffin embedded tissues that were stained for TRAP and Methyl green (Vector Laboratories). Osteoclast cells were identified as multinucleated TRAP-positive cells adjacent to bone. The measurement terminology and units used for histomorphometric analysis were those recommended by the Nomenclature Committee of the American Society for Bone and Mineral Research (Parfitt A, Drezner M K, Vlorieux F H, Kanis J A, Malluche H, Meunier P J, Ott S M, Recker R R. Bone histomorphometry:stardization of nomenclature, symbols, and units. Report of the ASBMR Histomorphometry Nomenclature Committee. J Bone Mineral Research 2, 595-610 (1987)).

### K/BXN Serum Transfer Arthritis Model

For arthritis experiments, K/BxN serum pools were prepared as described previously (Korganow A S, Weber J C, Martin T. [Animal models and autoimmune diseases]. Rev Med Interne 20, 283-286 (1999)). Arthritis in 8-week-old C57BL/6J male mice (The Jackson Laboratory) was induced by intraperitoneal injection of 100 μl of K/BxN serum on days 0 and 2. To analyze the effect of MDL28170 and CGP57380, the mice were randomized and treated with either vehicle (n=10), MDL28170 (10 mg/kg) or CGP57380 (40 mg/kg) with intraperitoneally (i.p) every day for 11 or 13 days. Vehicle or MDL28170 were prepared in 2.5% DMSO and 10% KLEPTOSE pH7.0 (Roquette Phama). CGP57380 was prepared in 4% DMSO and 30% PEG300 (Selleckchem) in 0.9% saline solution (BD science) (Lim S, et al. Targeting of the MNK-eIF4E axis in blast crisis chronic myeloid leukemia inhibits leukemia stem cell function. Proceedings of the National Academy of Sciences of the United States of America 110, E2298-2307 (2013)). The development of arthritis was monitored by measuring the thickness of wrist and ankle joints using dial-type calipers (Bel-Art Products) and scoring the wrist and ankle joints. For each animal, joint thickness was calculated as the sum of the measurements of both wrists and both ankles. Joint thickness was represented as the average for every treatment group. The severity of arthritis was scored in a blinded fashion by four investigators for each paw on a 3-point scale, in which 0=normal appearance, 1=localized edema or erythema over one surface of the paw, 2=edema or erythema involving more than one surface of the paw, 3=marked edema or erythema involving the whole paw. The scores of all four paws were added for a composite score (Murata K, et al. Hypoxia-Sensitive COMMD1 Integrates Signaling and Cellular Metabolism in Human Macrophages and Suppresses Osteoclastogenesis. Immunity 47, 66-79 e65 (2017)).

### Quantification and Statistical Analysis

Graphpad Prism 8.0 for Windows was used for all statistical analysis. Detailed information about statistical analysis, including tests and values used, and number of times experiments were repeated is provided in the figure legends. P values are provided in the text or the figure legends. Shapiro-Wilk normality tests were performed and for data that fell within Gaussian distribution, we performed appropriate parametric statistical tests and for those that did not fall within equal variance-Gaussian distribution, we performed appropriate non-parametric statistical tests. P<0.05 (*) was taken as statistically significant. Sample sizes were chosen according to standard guidelines. Number of animals was indicated as “n.”

### Example 2—Results

### Synovial CD14+ Cells Show a Distinct c-FMS Expression Pattern

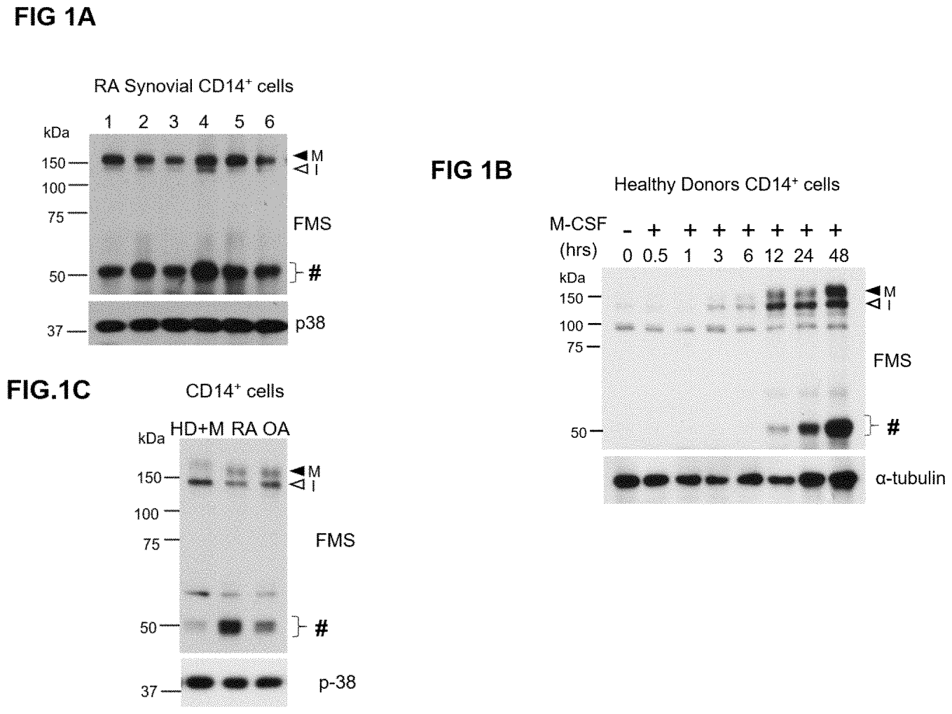

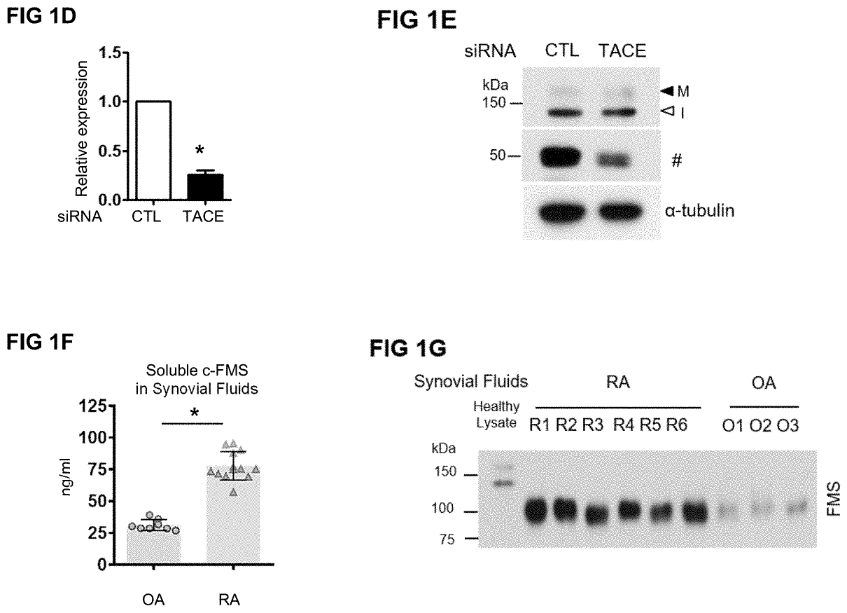

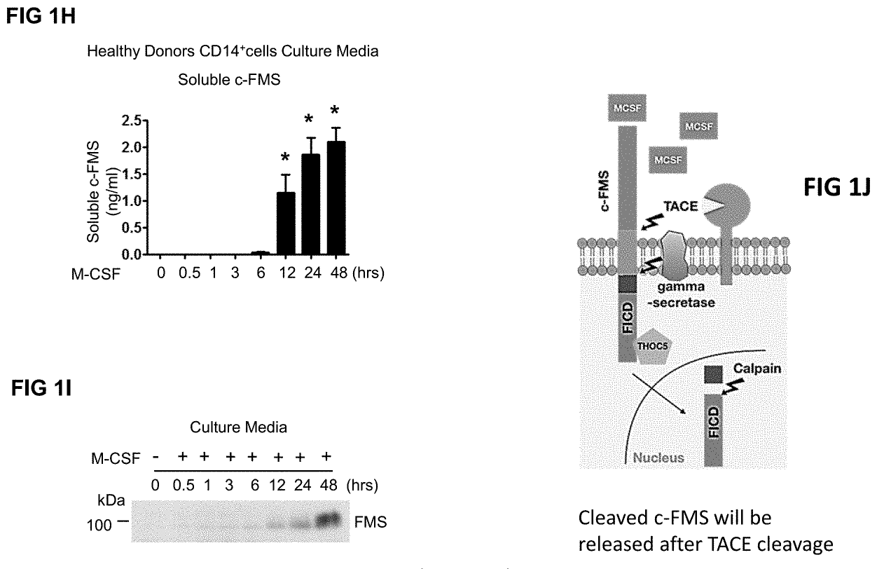

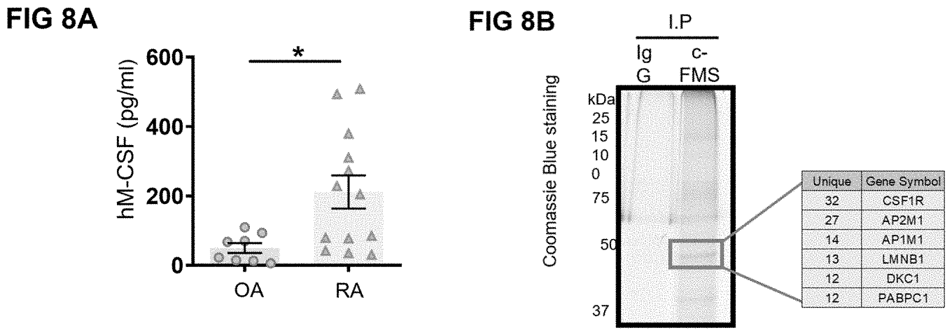

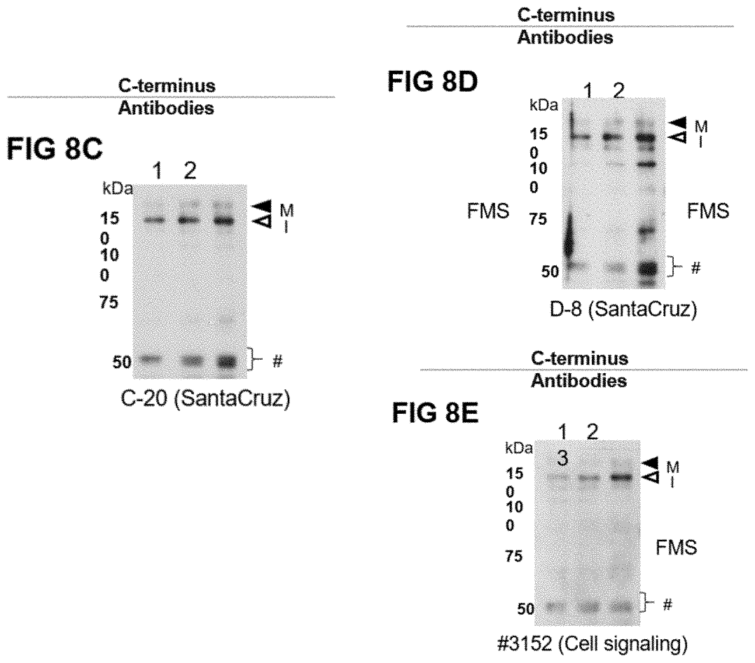

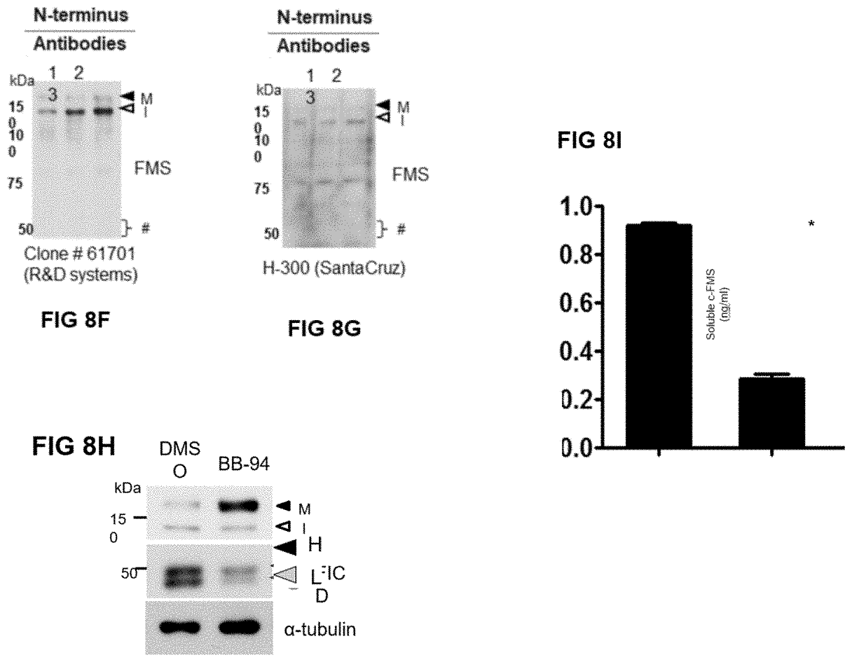

M-CSF/c-FMS signaling is implicated in the pathogenesis of RA. Consistent with a previous report showing increased M-CSF expression in RA synovial fluids (Paniagua, R. T. et al. c-Fms-mediated differentiation and priming of monocyte lineage cells play a central role in autoimmune arthritis. Arthritis Res Ther 12, R32, doi:10.1186/ar2940 (2010)), M-CSF levels were significantly higher in RA synovial fluids compared with osteoarthritis (OA) synovial fluids (FIG. 8A). We also measured the expression of cell-associated c-FMS in synovial CD14+ cells from RA patients. A c-FMS antibody against the C-terminal region of the receptor detected mature, glycosylated c-FMS (150 kDa, M), and immature, unglycosylated c-FMS (130 kDa, I) as expected. Intriguingly, we also detected small fragments of approximately 50 kDa in synovial CD14+ cells using anti-c-FMS antibodies (FIG. 1A). We next tested if CD14+ cells from healthy donors expressed small fragments. Immunoblot of freshly isolated CD14+ cells showed low levels of mature and immature c-FMS, but the small fragments were hardly detectable (FIG. 1B). After culturing fresh CD14+ cells with M-CSF, amounts of mature and immature c-FMS and small fragments increased in a time-dependent manner (FIG. 1B). When we compared the c-FMS expression between freshly isolated RA synovial CD14+ cells, M-CSF cultured CD14+ cells from healthy donors, and OA synovial CD14+ cells, we found higher levels of the small fragments in RA synovial CD14+ cells, while the levels of mature and immature c-FMS were comparable (FIG. 1C). To test whether the observed 50 kDa bands originated from c-FMS, mass spectrometry analysis was performed on the 50 kDa gel bands after immunoprecipitation with a c-FMS C-terminal antibody. Indeed, c-FMS was detected as the top ranked protein in the 50 kDa gel by mass spectrometry (FIG. 8B). To corroborate our findings, we tested if commercially available anti-c-FMS antibodies could detect the small fragments. The 50 kDa small fragments were detected by all antibodies against the C-terminal region of c-FMS (FIG. 8, C-E). However, they were not detected by antibodies against the N-terminal region of c-FMS (FIG. 8, F and G). These results suggest that the 50 kDa bands contained C-terminal regions of c-FMS. We named these 50 kDa bands ‘c-FMS intracellular cytoplasmic domains (FICDs)’.

To test if FICDs were generated by c-FMS proteolysis, OCP cells were prepared by culturing freshly isolated CD14+ cells with M-CSF to induce RANK expression, and TACE expression was knocked down using short interfering RNAs (siRNAs) in OCPs. TACE was decreased by 75% by TACE siRNA compared with control siRNA (FIG. 1D). As a result, TACE-knock down diminished the generation of FICDs upon M-CSF stimulation (FIG. 1E). Accordingly, the treatment with BB94, an MMP inhibitor, also suppressed the generation of FICDs (FIG. 8H) and inhibited the ectodomain shedding of c-FMS (FIG. 81). These results suggested that TACE cleavage was required for the generation of FICDs. To test if increased FICDs in RA synovial CD14+ cells were correlated with the shedding of c-FMS, we measured the level of soluble c-FMS in RA and OA synovial fluids. Soluble c-FMS was detectable by ELISA and immunoblot, and the level of soluble c-FMS was higher in RA synovial fluid than in OA synovial fluid (FIG. 1F). In addition, the soluble c-FMS in synovial fluids had a smaller molecular weight than full-length c-FMS (FIG. 1G), supporting that c-FMS proteolysis could be active in RA synovium. Accordingly, soluble c-FMS was not detected in freshly isolated CD14+ cells from healthy donors, but soluble c-FMS secretion in media gradually increased by culturing with M-CSF (FIG. 1, H and I).

### M-CSF Mediates the Generation of FICDs

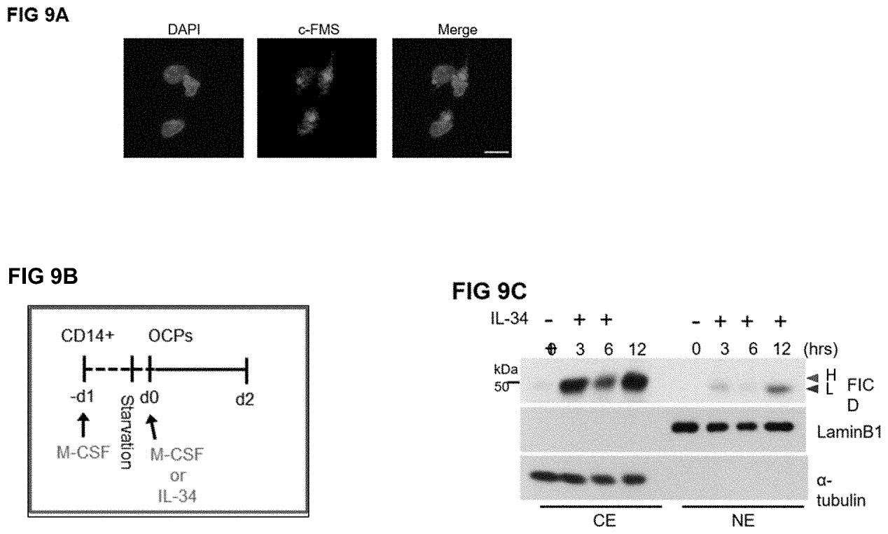

Consistent with the previous reports (Park-Min, K. H. Mechanisms involved in normal and pathological osteoclastogenesis. Cell Mol Life Sci 75, 2519-2528, doi:10.1007/s00018-018-2817-9 (2018).; Gebauer, F. & Hentze, M. W. Molecular mechanisms of translational control. Nat Rev Mol Cell Biol 5, 827-835, doi:10.1038/nrm1488 (2004)), c-FMS proteolysis was initiated by TACE (FIG. 1). We reasoned that FICD generation was followed a conventional RIPping process by ADAM family proteins and [symbol] -secretase (Kuhnle, N., Dederer, V. & Lemberg, M. K. Intramembrane proteolysis at a glance: from signalling to protein degradation. J Cell Sci 132, doi:10.1242/jcs.217745 (2019).). OCPs were treated with DAPT, a small molecule inhibitor of [symbol] -secretase (Lanz, T. A. et al. The gamma-secretase inhibitor N-[N-(3,5-difluorophenacetyl)-L-alanyl]-S-phenylglycine t-butyl ester reduces A beta levels in vivo in plasma and cerebrospinal fluid in young (plaque-free) and aged (plaque-bearing) Tg2576 mice. J Pharmacol Exp Ther 305, 864-871, doi:10.1124/jpet.102.048280 (2003)) and were fractionated into three categories: membrane, cytoplasm, and nucleus. The membrane-bound form had the highest molecular weight of FICD (mem), followed by the slightly smaller cytoplasmic FICD that was denoted high molecular mass FICD (H-FICD). Both forms were found to be larger than nuclear FICD, which was denoted L-FICD for low molecular mass FICD. Indeed, when we inhibited γ-secretase by the treatment with DAPT, membrane-bound FICD accumulated. However, we found cytosol and nuclear FICDs that were suppressed by DAPT (FIG. 2A). To further confirm the cellular localization of c-FMS and FICDs, we performed immunocytochemistry using the C-terminal region of a c-FMS antibody in human OCPs, and signals were detected by fluorescence and confocal microscopy analysis. Consistent with immunoblot analysis, positive signals of c-FMS were detected in the membrane (locus for mature form and mem), Golgi (locus for immature form), cytoplasm (locus for H-FICD), and nucleus (locus for L-FICD) (FIG. 2B and FIG. 9A). Since c-FMS signaling was required for FICD generation, we also tested if c-FMS signaling contributes to cellular localization of FICDs. Both M-CSF and IL-34, ligands for c-FMS21, induced the generation of H-FICD and L-FICD and cellular distribution of FICDs (FIG. 2C and FIG. 9B and C). OCPs treated with imatinib mesylate, an inhibitor of c-FMS activity, or with a c-FMS blocking antibody, suppressed not only FICD generation but also the levels of nuclear FICDs (FIG. 2D and E). Taken together, our results established that M-CSF/c-FMS signaling positively regulates the generation and cellular localization of FICDs in OCPs.

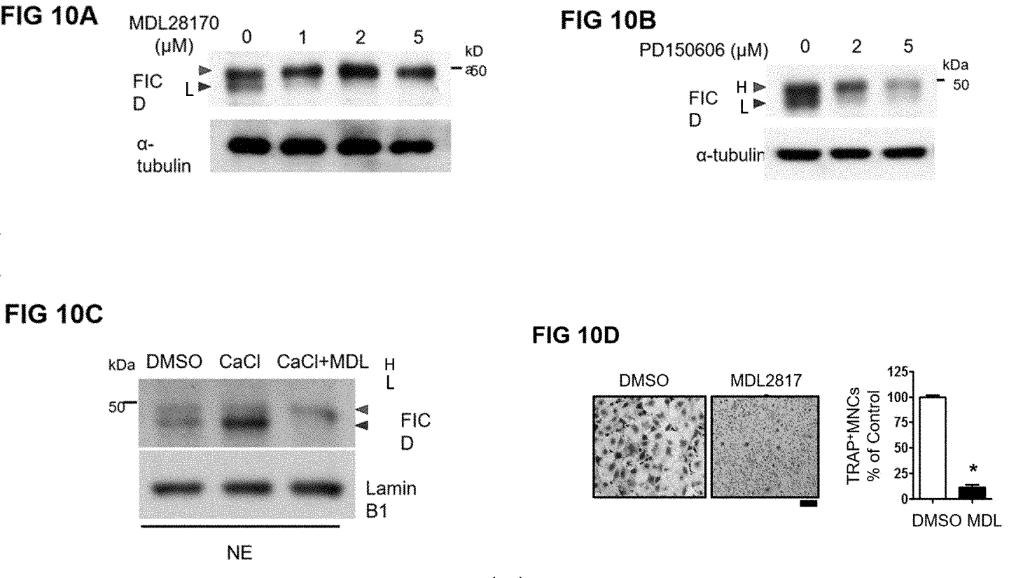

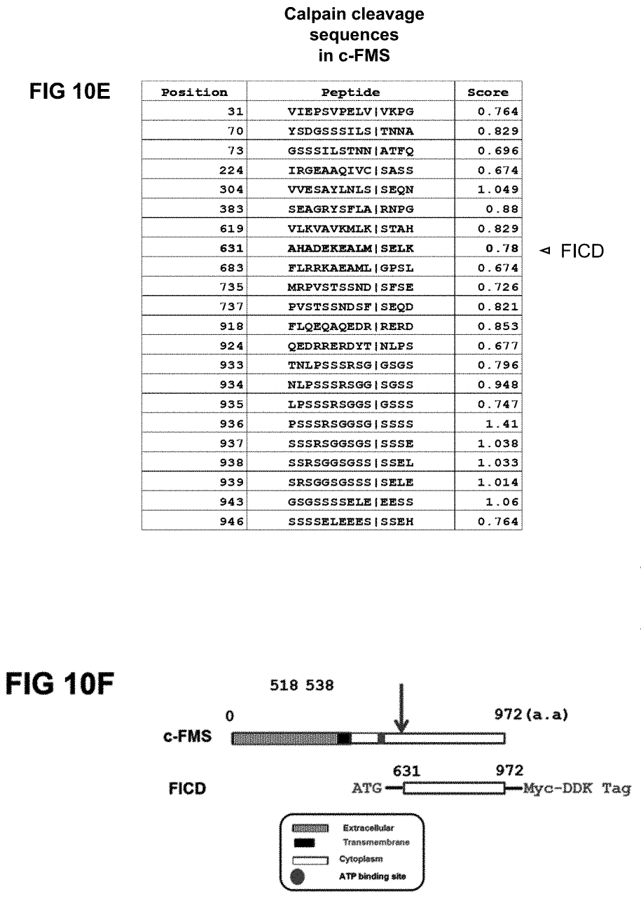

These results suggest that an additional protease may cleave H-FICD to become L-FICD. To identify the protease(s) responsible for cleavage of H-FICD in an unbiased manner, we performed a screening of 53 protease inhibitors using a protease library. The best hits associated with the inhibition of L-FICD generation were MDL28170 and PD150606—two calpain inhibitors—along with MMP inhibitors and γ-secretase inhibitors. Calpain is the family of calcium-dependent cytosolic cysteine proteases expressed ubiquitously in mammals and many other organisms (Pfaff, M., Du, X. & Ginsberg, M. H. Calpain cleavage of integrin beta cytoplasmic domains. FEBS Lett 460, 17-22 (1999)), and calpain-dependent cleavages contribute to modulating various cellular functions such as apoptosis, proliferation and migration (Deshpande, R. V. et al. Calpain expression in lymphoid cells. Increased mRNA and protein levels after cell activation. J Biol Chem 270, 2497-2505 (1995). Svensson, L. et al. Calpain 2 controls turnover of LFA-1 adhesions on migrating T lymphocytes. PloS one 5, e15090, doi:10.1371/journal.pone.0015090 (2010).). Calpain has been implicated to be important for osteoclastogenesis and migration (Marzia, M. et al. Calpain is required for normal osteoclast function and is down-regulated by calcitonin. J Biol Chem 281, 9745-9754, doi:10.1074/jbc.M513516200 (2006). Yaroslayskiy, B. B., Sharrow, A. C., Wells, A., Robinson, L. J. & Blair, H. C. Necessity of inositol (1,4,5)-trisphosphate receptor 1 and mu-calpain in NO-induced osteoclast motility. J Cell Sci 120, 2884-2894, doi:10.1242/jcs.004184 (2007).), although the exact mechanisms and targets of calpain are unknown. We found that inhibiting calpain suppressed the generation of FICD in a dose-dependent manner (FIG. 10A and B). The inhibition of calpain by MDL28170 did not interfere with the translocation of FICD into the nucleus but instead shifted the enrichment of L-FICD to H-FICD in the nucleus, suggesting that calpain cleavage may occur in the nucleus (FIG. 2F). Since calpain is activated by calcium, we examined if calcium signaling could compensate for M-CSF signaling to generate FICDs. In the absence of c-FMS signaling, calcium signaling was able to promote the cleavage of L-FICD to H-FICD in the nucleus, and MDL29170 reversed the effect of calcium signaling on L-FICD processing (FIG.10C). Consistent with the previous results, the treatment with MDL28170 decreased not only L-FICD but also osteoclast differentiation (FIG. 10D).

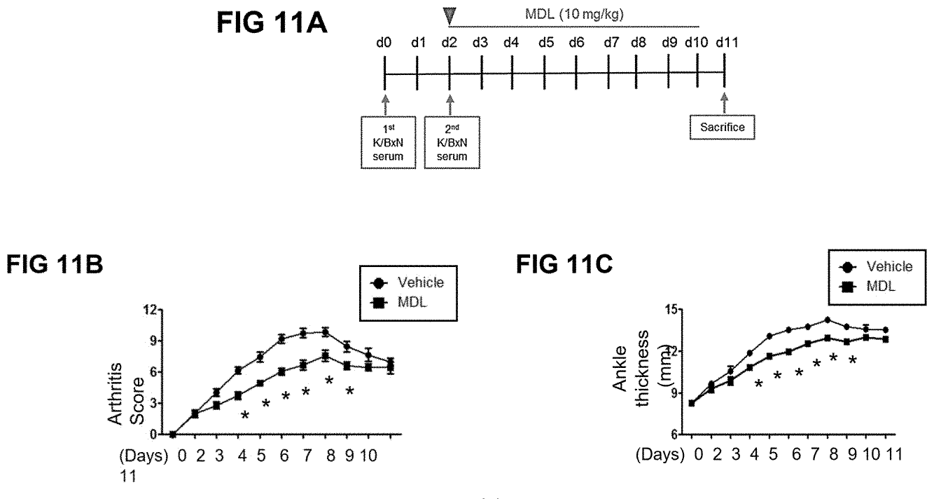

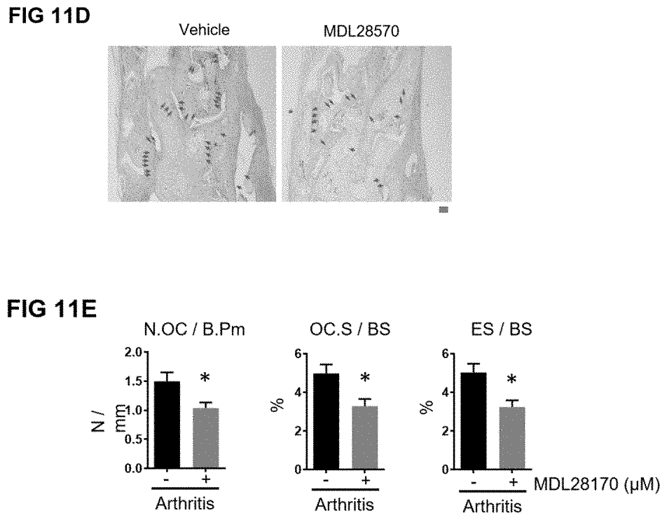

To further investigate which form of calpain cleaves FICD in the nucleus, we used siRNAs to knock downed Calpain 1, 5, and 6, which are expressed in OCPs. Calpain 1, 5, and 6 were efficiently knock downed (KD) using siRNAs (FIG. 2G). Among them, Calpain 1 KD cells were unable to process H-FICD to L-FICD, resulting in H-FICD accumulation in the nucleus of calpain 1 KD cells (FIG. 2H). Thus, our results reveal that calpain 1 plays a key role in proteolysis of H-FICD to L-FICD. To address the (patho)physiological importance of FICD in inflammatory bone erosion, we tested the effect of MDL28170 on bone erosion in a K/BxN serum transfer induced arthritis. K/BxN serum was administrated intra-peritoneally on day 0 and day 2, and then, MDL28170 was administrated after disease onset (FIG. 11A). The severity of arthritis was assessed by a clinical score and ankle joint thickness, which were attenuated by MDL28170 treatment (FIG. 11B and C). The treatment with a calpain inhibitor decreased the number of osteoclasts and attenuated bone erosion in a K/BXN serum-induced arthritis model (FIG. 11D and E). Taken together, our results suggest that c-FMS proteolysis generate FICD by sequential proteolysis by TACE, [symbol] -secretase, and calpain 1.

### Blocking c-FMS Proteolysis Suppresses RANKL-Induced Osteoclast Formation and Activity

Given that FICD levels were higher in RA synovial CD14+ cells and administration of a calpain inhibitor suppressed both inflammation and bone erosion, we hypothesized that c-FMS proteolysis plays an important role in macrophage functions including inflammatory responses and osteoclastogenesis. To test our hypothesis, we generated non-cleavable c-FMS mutants by mutating TACE cleavage sites (named Fmsmut, FIG. 3A) that could not produce FICDs (Vahidi, A., Glenn, G. & van der Geer, P. Identification and mutagenesis of the TACE and gamma-secretase cleavage sites in the colony-stimulating factor 1 receptor. Biochemical and biophysical research communications 450, 782-787, doi:10.1016/j.bbrc.2014.06.061 (2014).). 293T cells that had no endogenous c-FMS expression were transduced with lentiviral particles encoding control, wild-type FMS (called FMSwt), or Fmsmut. Cell surface expression of both FMSwt and Fmsmut was detected by flow cytometry analysis, and Fmsmut was resistant to TPA-induced TACE-mediated shedding compared with FMSwt (FIG. 12A). Importantly, when cells were stimulated with M-CSF, the activation of ERK, JNK, and p38 by Fmsmut was comparable to that of FMSwt (FIG. 3B), suggesting that Fmsmut is a functional receptor. To minimize the effect of the endogenous FICD, we also used bone marrow derived macrophages (BMDMs) from c-FMS inducible conditional haplodeficient mice (c-FMS f/+ΔMX1) that were generated by crossing c-FMS floxed mice with MX1 cre mice 27. FMS haplodeficient BMDMs from FMSf/+MX1cre mice were transduced with lentiviral particles encoding control, FMSwt or Fmsmut. Since c-FMS haplodeficient BMDMs expressed a low level of FICD (FIG. 12B), endogenous FICD was detected in Fmsmut transduced cells (FIG. 12C). As expected, FICD generation in both Fmsmut transduced FMS haplodeficient BMDMs and 293T cells was diminished compared to FMSwt transduced cells (FIG. 9, C and D). To test the effect of c-FMS proteolysis on inflammatory responses, control, FMSwt, and Fmsmut transduced cells were stimulated with LPS, a Toll-like receptor 4 (TLR4) agonist, and we measured the expression of pro-inflammatory cytokines such as TNFα and IL6. The expression of TNFα, and IL6 mRNA was induced by LPS and was comparable among the groups (FIG. 3C). Consistently, the production of TNFα and IL6 protein was also comparable between FMSwt and Fmsmut transduced cells (FIG. 3D). These results suggest that FICDs may have a minimal effect on inflammation. Next, we tested the role of c-FMS proteolysis in the osteoclastogenic responses to the TNF family cytokine RANKL. M-CSF signaling is a key regulator of osteoclast differentiation (Tsukasaki, M. & Takayanagi, H. Osteoimmunology: evolving concepts in bone-immune interactions in health and disease. Nat Rev Immunol 19, 626-642, doi:10.1038/s41577-019-0178-8 (2019)). As expected, ectopic expression of FMSwt enhanced osteoclast differentiation and bone resorbing activity compared with control cells (FIG. 3E). Strikingly, ectopic expression of Fmsmut showed diminished osteoclast formation relative to that of FMSwt-expressing cells (FIG. 3E), indicating that Fmsmut could not efficiently promote osteoclastogenesis like FMSwt. Concomitantly, the increased bone resorbing activity of FMSwt-expressing cells was also diminished in FMSmut-expressing cells (FIG. 3F). Therefore, our results suggested that increased FICD levels in FMSwt-expressing cells contribute to osteoclast differentiation and activity while having no effect on inflammatory responses.

### FICD Knock-In Mice Exhibit Osteoporotic Phonotype

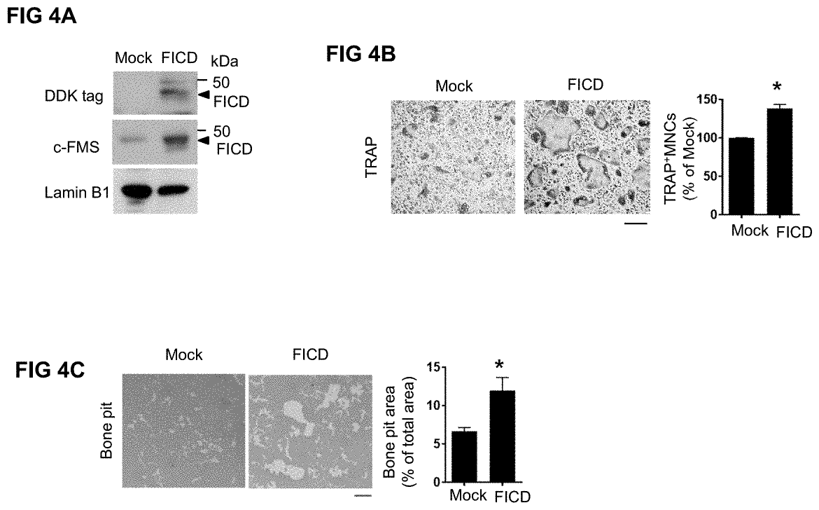

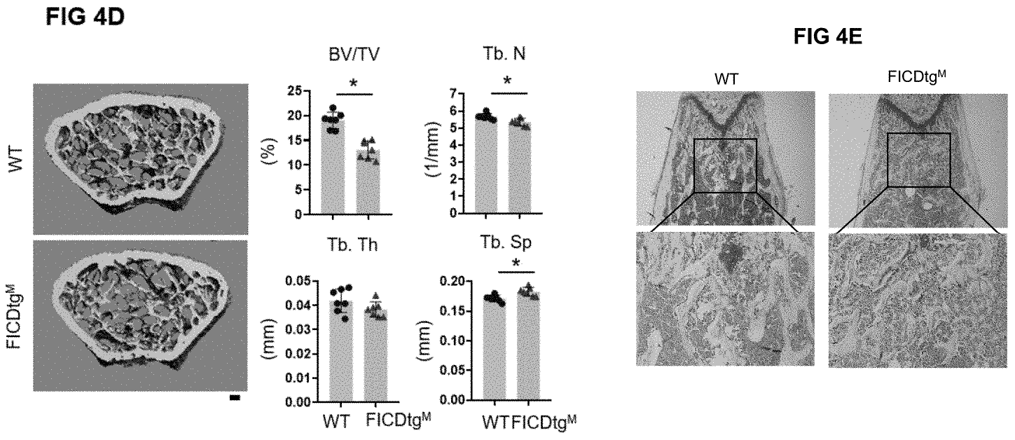

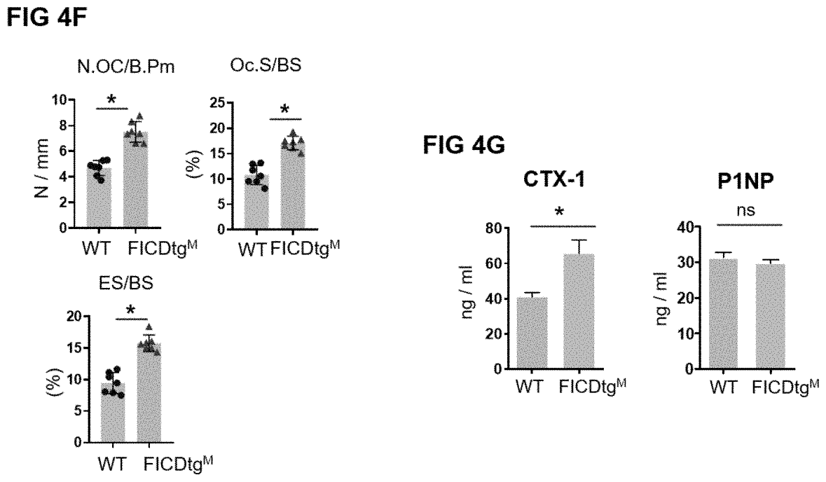

To further delineate the role of FICD in osteoclasts, DDK-tagged FICD was generated based on N-terminal sequencing and predicted protease cleavage sites (FIG. 10E, F). BMDMs were transduced by retroviral particles encoding DDK-tagged FICD. FICD protein expression increased in FICD-transduced cells (FIG. 4A). Ectopic FICD expression enhanced RANKL-induced osteoclast differentiation and resorption when compared with control cells (FIG. 4B and C), suggesting that constitutive FICD expression promotes osteoclasatogenesis.

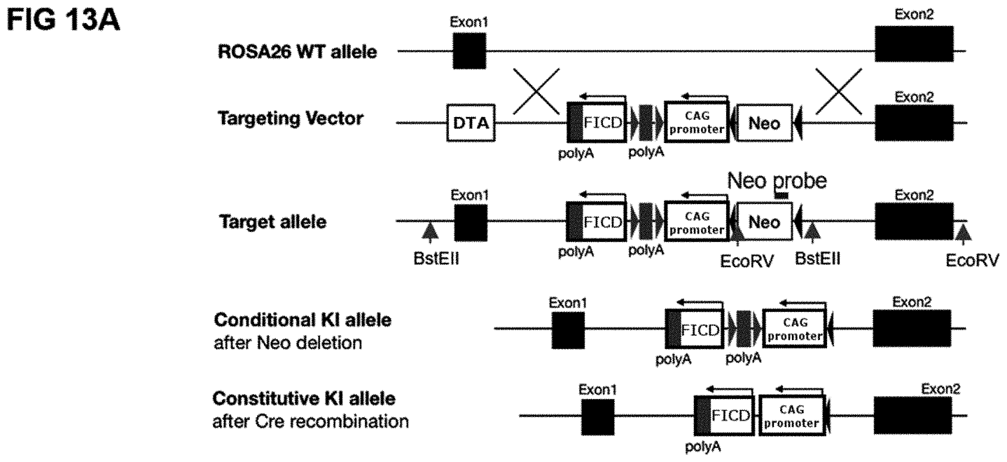

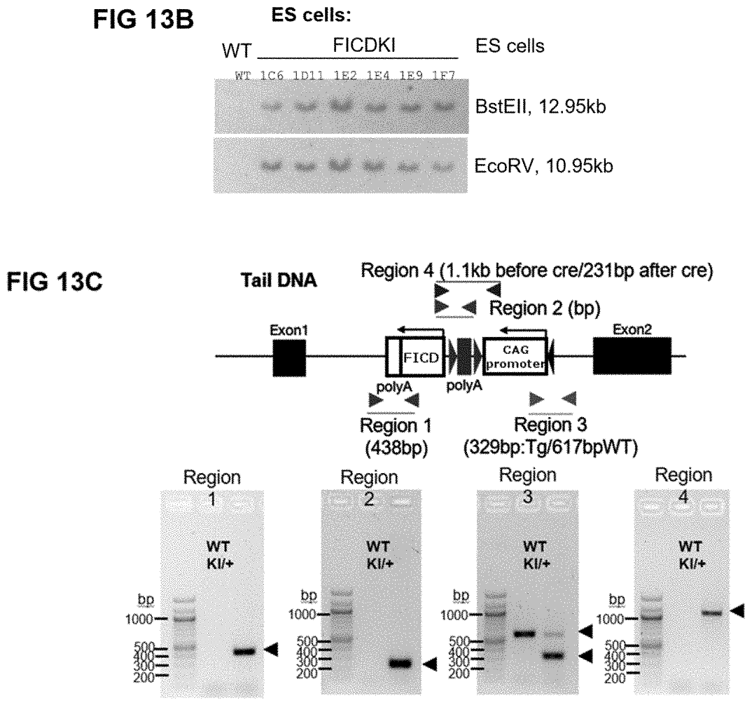

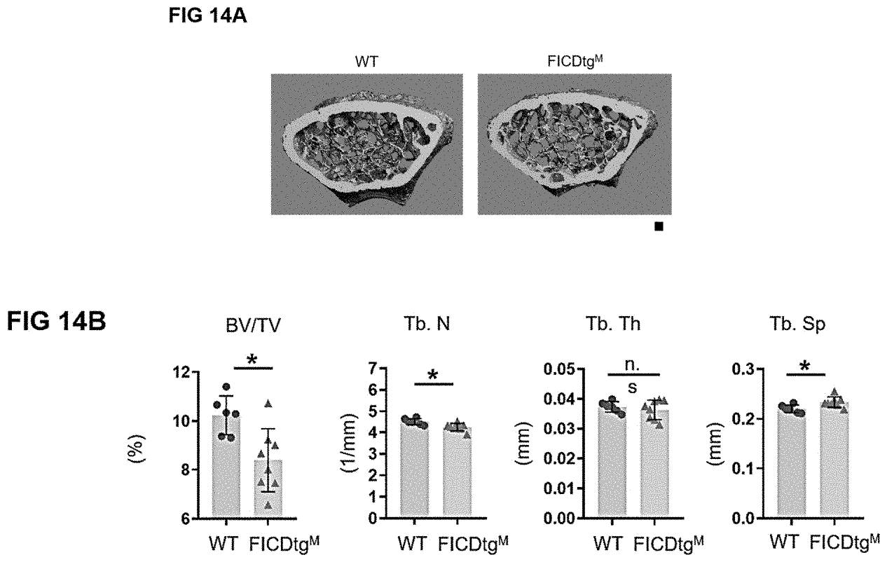

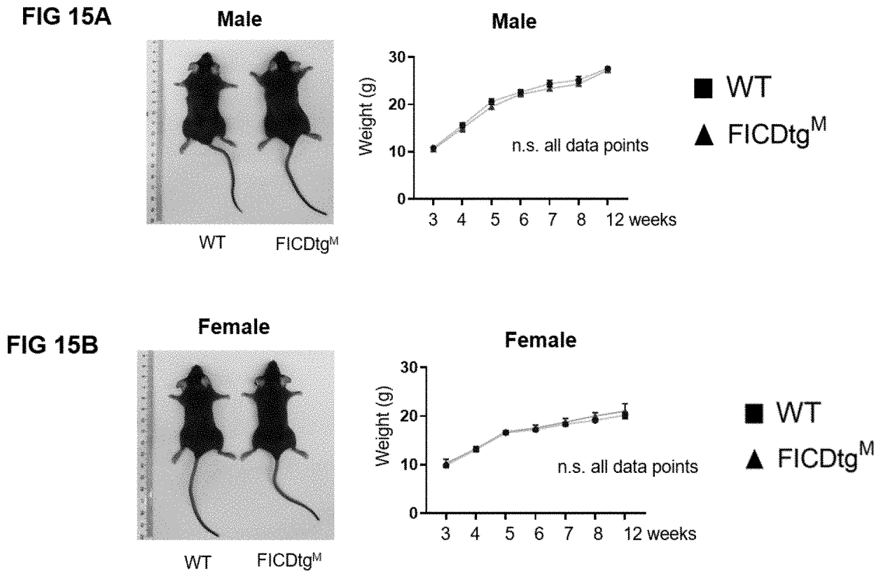

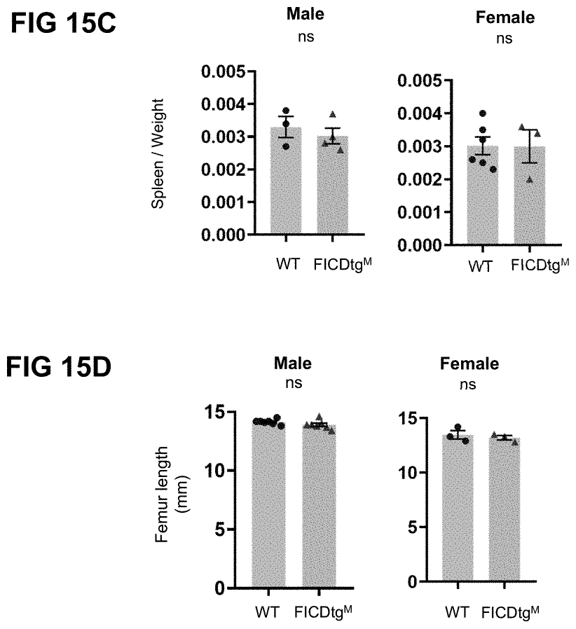

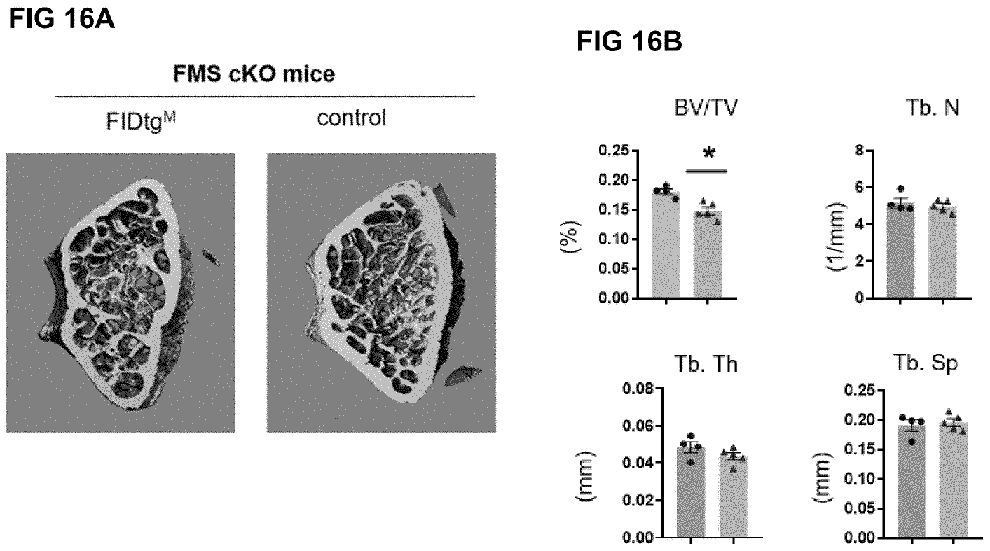

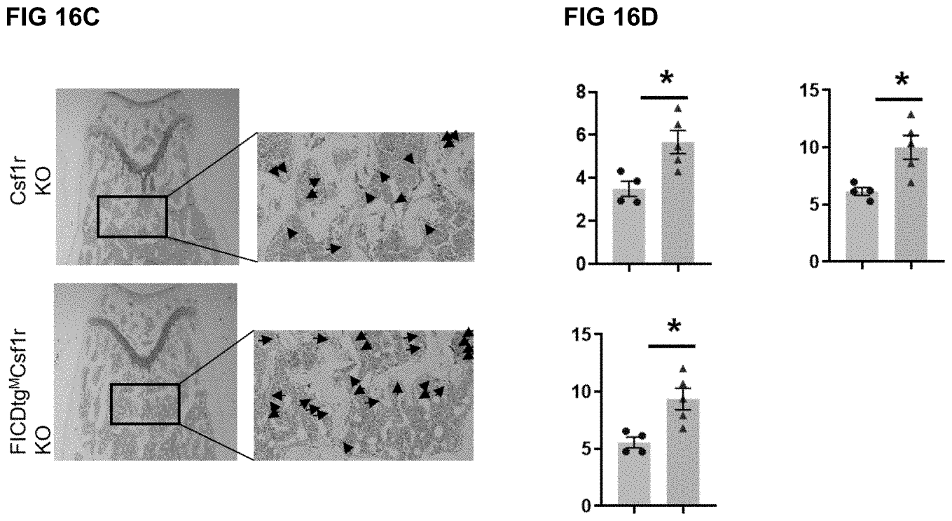

Increased FICD expression was observed in macrophages from RA patients (FIG. 1). To model the high expression of FICD in vivo, we generated myeloid cell-specific conditional FICD knock-in mice, FICDKI/KI×Lyz2-crehet mice, by crossing FICDKI/KI with myeloid cell specific LysM-driven CRE recombinase (referred to as FICDtg^{M}; FIG. 13). FICD expression was detected by immunoblot using anti-HA antibodies and effectively increased in BMDMs (FIG. 4D). We tested if FICD regulates in vivo osteoclastogenesis. In micro-CT analysis, FICDtg^{M} male and female mice exhibited decreased bone mass, where bone volume/tissue volume (BV/TV) ratio and trabecular number (Tb.N) were significantly decreased compared with control mice (FIG. 4E and FIG. 14A and B). Histomorphometry analysis also showed that the number of osteoclasts, osteoclast surface area, and eroded surfaces were significantly higher in FICDtg^{M} mice than in control LysM cre (WT) mice (FIG. 4F and G). Accordingly, serum CTX was higher in FICDtg^{M} mice relative to control mice while P1NP level was similar between control and FICDtg^{M} mice (FIG. 4H). However, overt phenotypes including body weight, spleen weight, and femur length were not different between control and FICDtg^{M} mice (FIG. 15A-D), suggesting that FICD overexpression in myeloid cells did not affect the gross phenotype. In addition, FICDtg^{M} mice in c-FMS null background also exhibited diminished bone mass compared control c-FMS null mice (FIG. 16A and B) and showed the increased in vivo osteoclast activity (FIG. 16B and D). Overall, our findings suggest that FICD expression in OCPs results in decreased bone mass by increasing osteoclasts under physiological conditions.

### FICD Accelerates Arthritis-Induced Bone Erosion

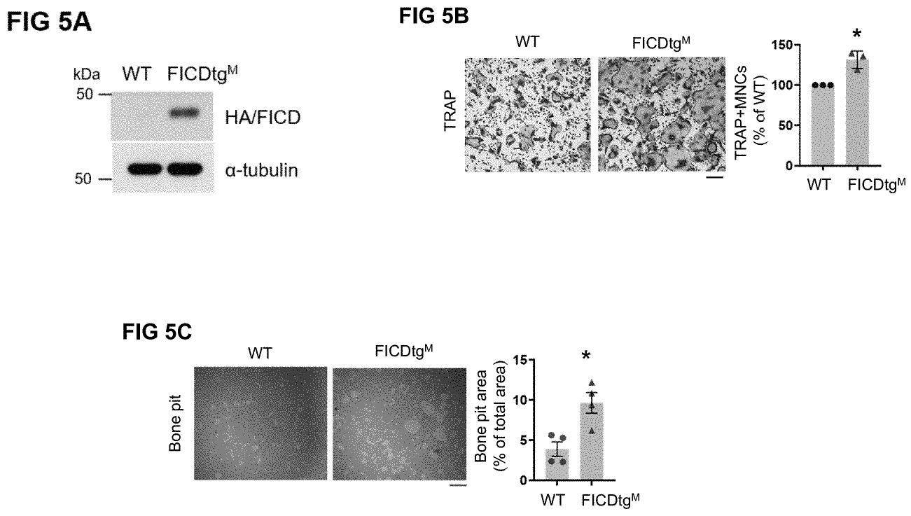

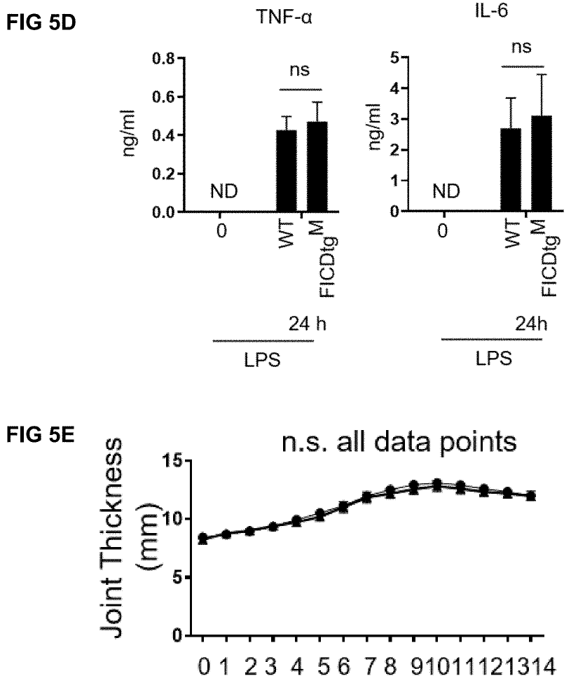

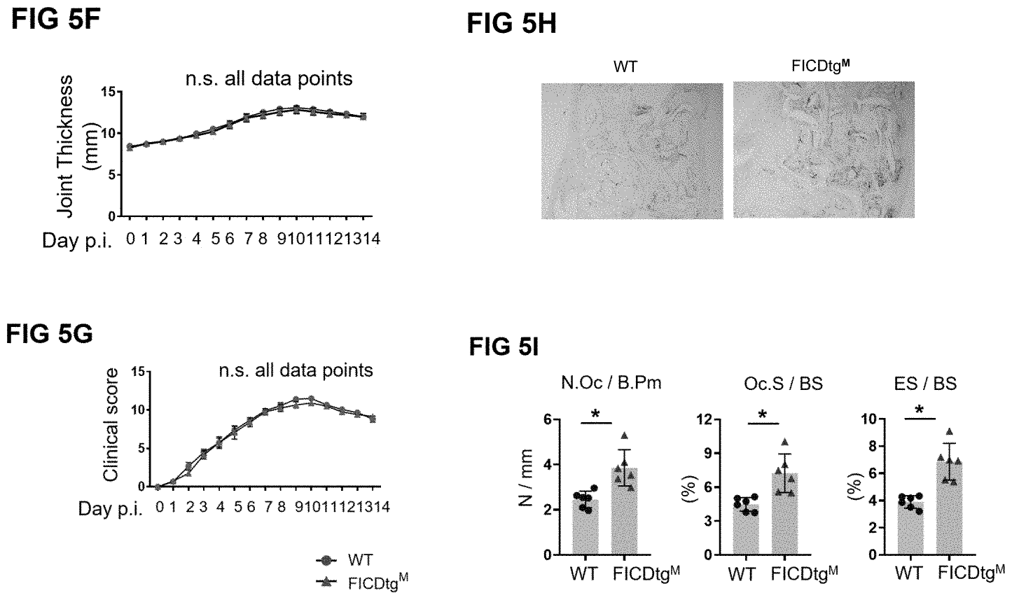

Given the high levels of FICD in synovial CD14+ cells and its positive regulation of osteoclastogenesis without any effect on inflammatory responses, we hypothesized that FICD may play a role in arthritic bone erosion. We first determined the effects of FICD on inflammation and osteoclast differentiation in vitro. BMDMs from FICDtg^{M} mice or WT mice were cultured with M-CSF and RANKL to form osteoclasts in vitro. Consistent with in vivo data, FICDtg^{M} cells showed significantly enhanced osteoclast differentiation and bone resorption activity relative to control cells (FIG. 5A and B). To measure the role of FICD in inflammation, OCPs were stimulated with LPS (10 ng/ml). mRNA and protein expression of LPS-induced TNFα and IL6 were comparable between FICDtg^{M} cells and control cells (FIG. 5C and D). To address the importance of FICD in osteoclast-mediated pathological bone resorption, we tested the effects of FICD on bone erosion in a murine K/BxN serum-transfer induced arthritis model (Kouskoff, V. et al. Organ-specific disease provoked by systemic autoimmunity. Cell 87, 811-822, doi:10.1016/s0092-8674(00)81989-3 (1996)). K/BxN serum was administered intra-peritoneally at 0 and 2 d, and the arthritis severity was assessed by a clinical score and ankle joint thickness until 14 d. FICDtg^{M} mice exhibited minimal differences in joint swelling or inflammation compared with littermate control mice in K/BxN serum-induced arthritis (FIG. 5E and F). However, histomorphometry analysis revealed that osteoclast number, osteoclast surface area, and eroded surface in periarticular bone of FICDtg^{M} mice were significantly increased compared to those of WT mice (FIG. 5G and H). Thus, our results suggest a promoting role of FICD in pathological bone loss under inflammatory conditions in vivo.

### FICD Regulates RANKL-Induced NFATc1 Expression via the MNK1/2/eIF4E Axis

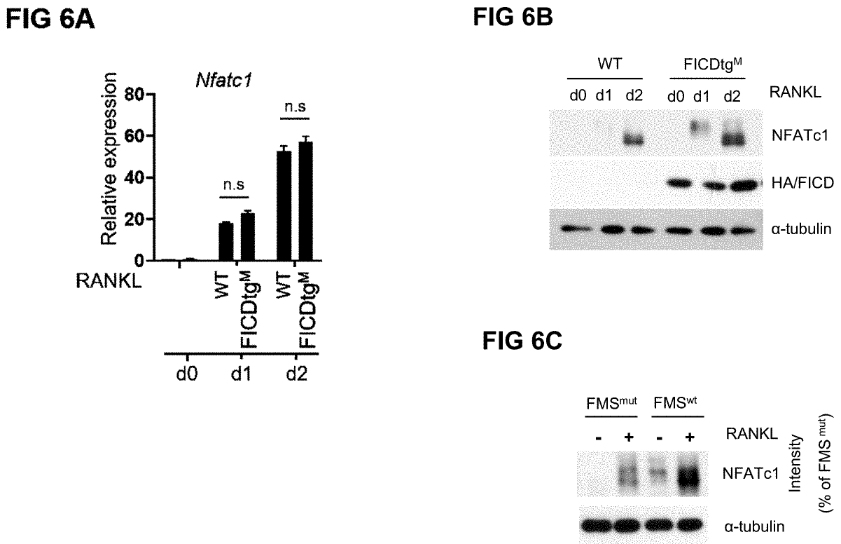

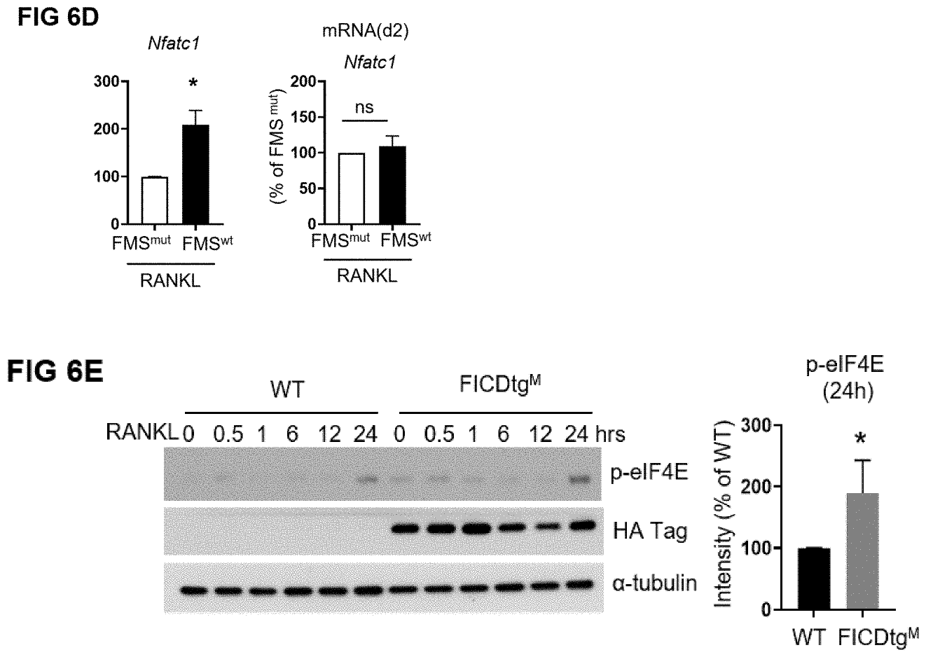

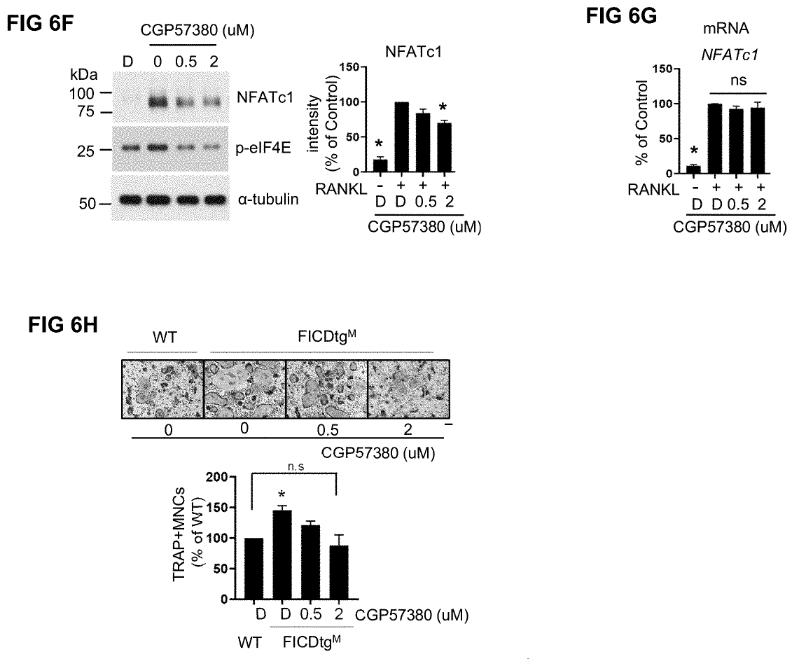

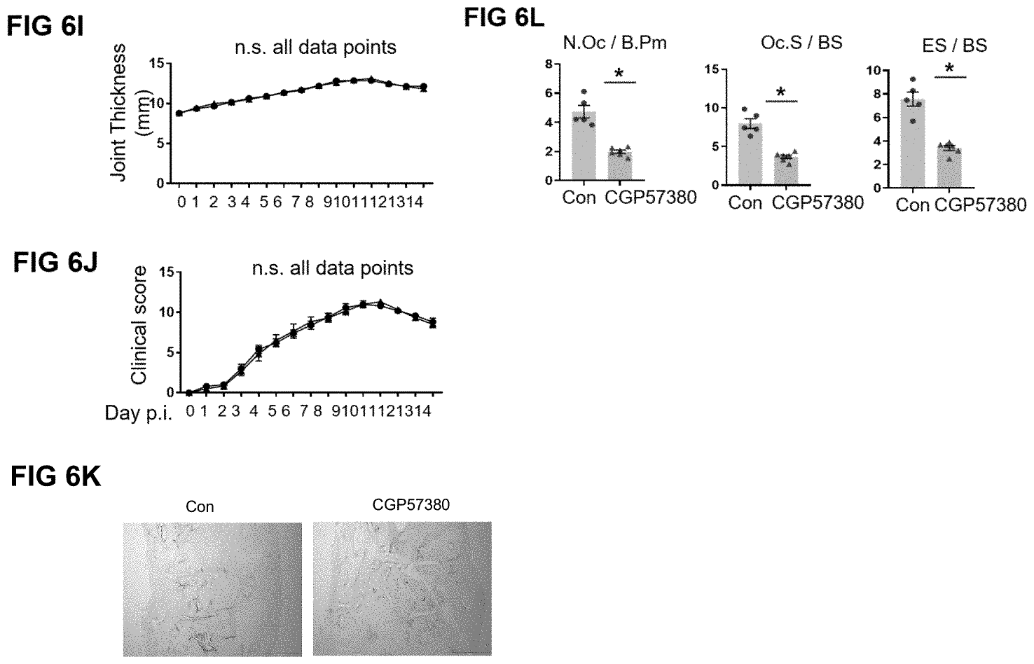

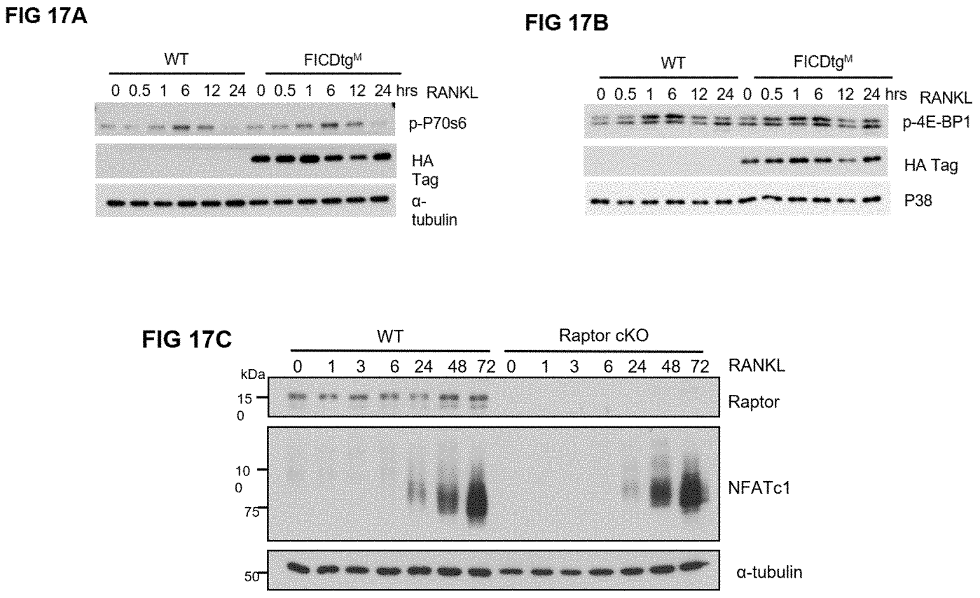

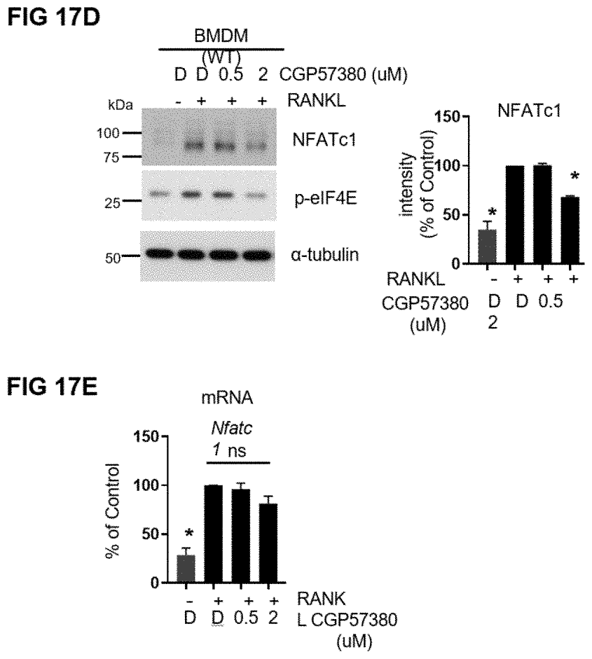

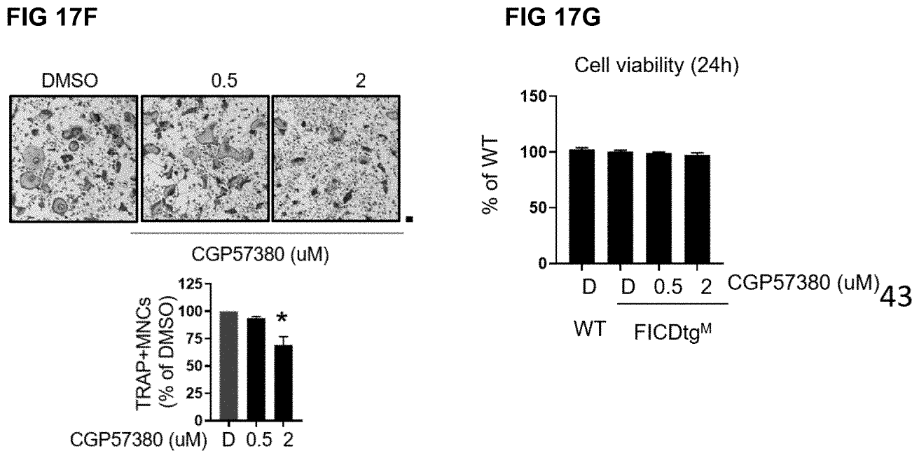

To gain insight into the mechanism by which FICDs regulate osteoclastogenesis, we tested the effect of FICD on the expression of NFATc1, a master regulator of osteoclastogenesis (Negishi-Koga, T. & Takayanagi, H. Ca2+-NFATc1 signaling is an essential axis of osteoclast differentiation. Immunol Rev 231, 241-256, doi:10.1111/j.1600-065X.2009.00821.x (2009)). Nfatc1 mRNA was comparable between WT and FICDtg^{M} mice (FIG. 6A). However, RANKL-induced NFATc1 protein levels were substantially increased in FICDtg^{M} cells compared with WT cells (FIG. 6B). Consistently, NFATc1 protein expression was diminished by impaired FICD generation in Fmsmut compared with FMSwt, while Nfatc1 mRNA expression was comparable between Fmsmut and FMSwt (FIG. 6C and D). To explain the considerable discrepancy in the Nfatc1 mRNA and protein levels between WT and FICDtg^{M} osteoclasts, we tested the effect of FICD on the activation of the mammalian target of the rapamycin (mTOR) pathway, and on the induction of the MAPK interacting kinases (MNK1/2)-dependent pathway among several key signaling pathways that regulate protein translation (Sonenberg, N. & Hinnebusch, A. G. Regulation of translation initiation in eukaryotes: mechanisms and biological targets. Cell 136, 731-745, doi:10.1016/j.ce11.2009.01.042 (2009).). We measured phospho-eIF4E as a downstream readout for the activation of the mTORC1 and MNK1/2 pathways. Strikingly, eIF4E phosphorylation was activated by RANKL stimulation and was significantly increased in FICDtg^{M} cells compared with WT cells (FIG. 6E), suggesting that FICD may enhance eIF4E-dependent protein synthesis. To further delineate the cause of increased eIF4E phosphorylation, we measured phospho-S6K and phospho-4EBP1 to determine the activation of mTORC1 pathway. RANKL-induced mTORC1 activation was comparable between FICDtg^{M} and control cells (FIG. 17A and B). Consistent with the literature (Huynh, H. & Wan, Y. mTORC1 impedes osteoclast differentiation via calcineurin and NFATc1. Commun Biol 1, 29, doi:10.1038/s42003-018-0028-4 (2018)), NFATc1 protein expression was comparable between control OCPs, and RAPTOR-deficient cells, a model for low mTORC1 signals (FIG. 17C). Our data suggest that the mTORC1 pathway unlikely regulates FICD-induced eIF4E phosphorylation. We next tested if the MNK1/2-eIF4E axis regulates RANKL-induced NFATc1 expression using an MNK1/2 inhibitor, CGP57380 32. Inhibiting MNK1/2 activity indeed suppressed RANKL-induced NFATc1 protein expression in a dose-dependent manner in both human and mouse OCPs, whereas Nfatc1 mRNA expression was marginally changed by the CGP57380 treatment (FIG. 6F and G; FIG. 16D and E). CGP57380 also suppressed osteoclast differentiation in a dose-dependent manner in BMDM cells (FIG. 16F). To test the contribution of the MNK1/2 pathway on increased osteoclastogenesis in FICDtg^{M} cells, we treated FICDtg^{M} cells with CGP57380. As expected, we found that suppressing MNK1/2/p-eIF4E inhibited the enhanced osteoclastogenesis in FICDtg^{M} cells to become comparable to osteoclasts in WT cells (FIG. 6H). However, CGP57380 treatment showed a minimal effect on cell viability (FIG. 61). We tested the effect of CGP57380 on bone erosion in a K/BxN serum transfer induced arthritis. K/BxN serum was administrated intra-peritoneally on day 0 and day 2, and then, CGP57380 was administrated after disease onset (FIG. 6J). The severity of arthritis was assessed by a clinical score and ankle joint thickness, which were not affected by MDL28170 treatment (FIG. 6K and L). The treatment with a MNK1/2 inhibitor decreased the number of osteoclasts and attenuated bone erosion in a K/BXN serum-induced arthritis model (FIG. 6M and N). Overall, our results suggest that increased phospho-eIF4E is a key regulator of increased osteoclastogenesis in FICDtg^{M} cells and targeting the FICD/MNK1/2 axis was significantly diminished arthritic bone erosion.

### FICD/DAP5/Fxr1 Complexes Activate the MNK1/2 Pathway and NFATc1 Expression

Next, we sought to identify the underlying mechanisms by which FICD increases eIF4E phosphorylation. We performed an unbiased proteomic analysis using mass spectrophotometry with two biological replicates to screen proteins that both interact with FICD and regulate the MNK1/2 pathway. FICD-DDK was transfected in 293T cells, and FICD interacting proteins were immunoprecipitated using anti-c-FMS antibodies. 145 FICD-interacting proteins were identified (Table 2, below).

TABLE 2
FICD-interacting proteins

| | | | | |
|---|---|---|---|---|
| CSF1R | PDHB | TBL2 | SPCS3 | TAF8 |
| NUP133 | PSMC3 | UFL1 | TAF5L | THOC5 |
| NUP107 | PSMC5 | XPNPEP3 | YARS2 | VAPB |
| NKRF | UFD1 | ARF4 | AAR2 | ABCF1 |
| NDUFS1 | ATP5O | BTAF1 | ANAPC1 | ATP5J2 |
| NUP98 | GTF3C3 | CBX8 | CCDC47 | AURKB |
| PSMD2 | U2SURP | DLAT | CLPB | BZW1 |
| MOGS | ATP5C1 | HLTF | CTNNBL1 | CDK1 |
| NDUFS2 | BPTF | MRPL13 | DCUN1D5 | CSNK1A1 |
| ERCC3 | DPM1 | NUP43 | DLST | EIF3F |
| LRPPRC | DRG1 | POLR1A | EIF3H | ERLIN2 |
| ATP5A1 | EIF4G2 | PRPF38A | GADD45GIP1 | EXOSC3 |
| AAAS | KPNA2 | SDCBP | GAPDH | FXR1 |
| DDX18 | LMAN2 | SUPV3L1 | GATAD2B | GPD2 |
| GTF2H4 | MTA1 | TRIP13 | GNAI3 | HARS |
| NUP155 | NEFL | ARL1 | GTF3C2 | KPNA5 |
| DDX1 | PDS5B | DDX6 | HSD17B10 | LEMD3 |
| IQGAP1 | PSMD6 | EIF3L | KIF11 | LGALS3BP |
| MTA2 | STRAP | EMC3 | KPNA3 | MCCC2 |
| PSMD1 | TUBGCP2 | MRPL3 | LUC7L3 | MRPL44 |
| SMARCD2 | XPO5 | MRPS31 | NDUFS8 | MRPL53 |
| DDB1 | ANAPC5 | MTDH | PELP1 | NDUFB4 |
| DNAJA1 | CDC73 | MTHFD1L | POP1 | NGDN |
| EFTUD2 | CFL1 | ORC4 | PPIE | NRAS |
| PSMD3 | GTF3C5 | POLDIP2 | PRKAG1 | PTRH2 |
| RPN2 | HSD17B12 | REXO4 | PSMB4 | RACGAP1 |
| ANAPC4 | HYOU1 | RFC1 | RARS | SRPK1 |
| CUL1 | POGZ | RFC3 | RPL32 | SUZ12 |
| NDUFA10 | PPAN | SEC63 | SIN3A | TSG101 |

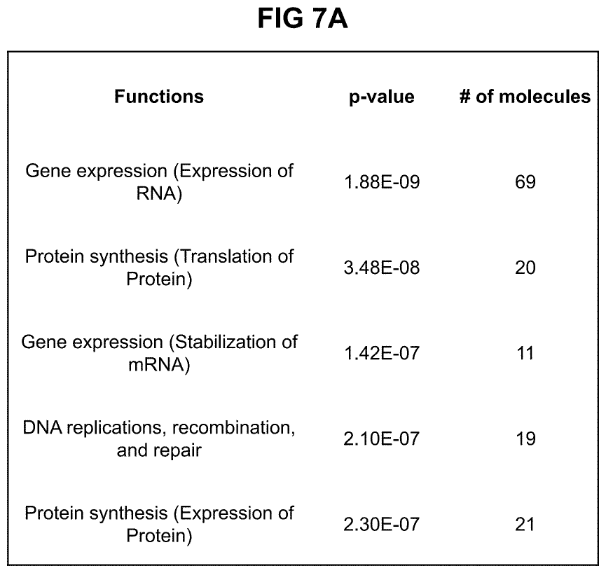

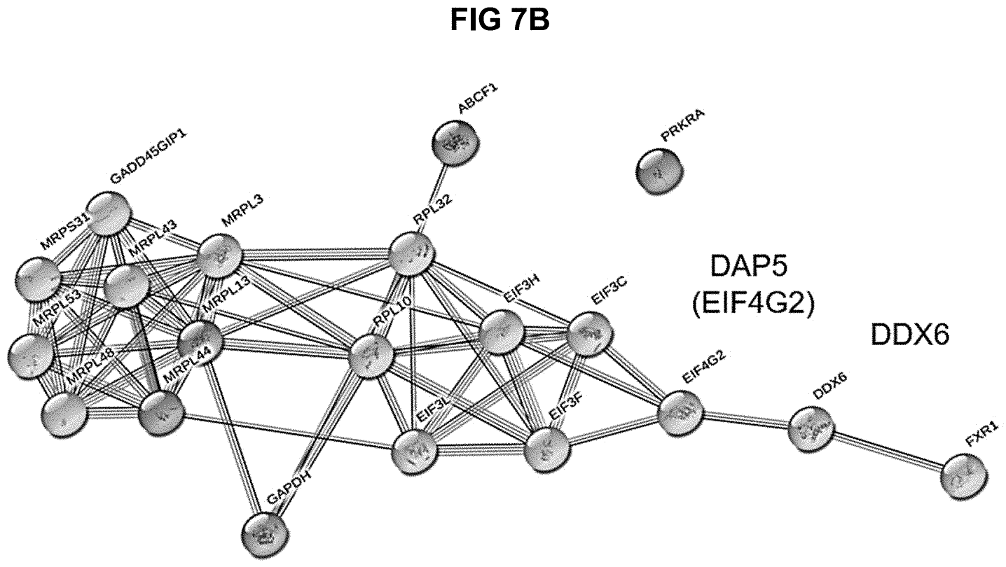

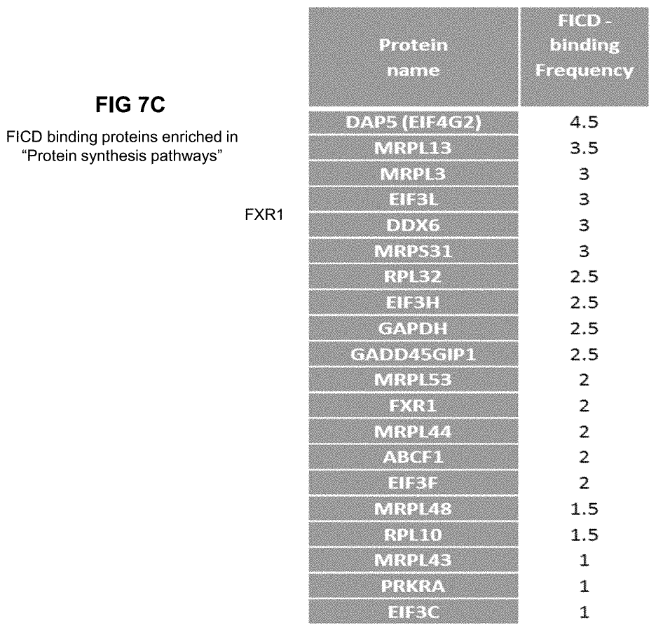

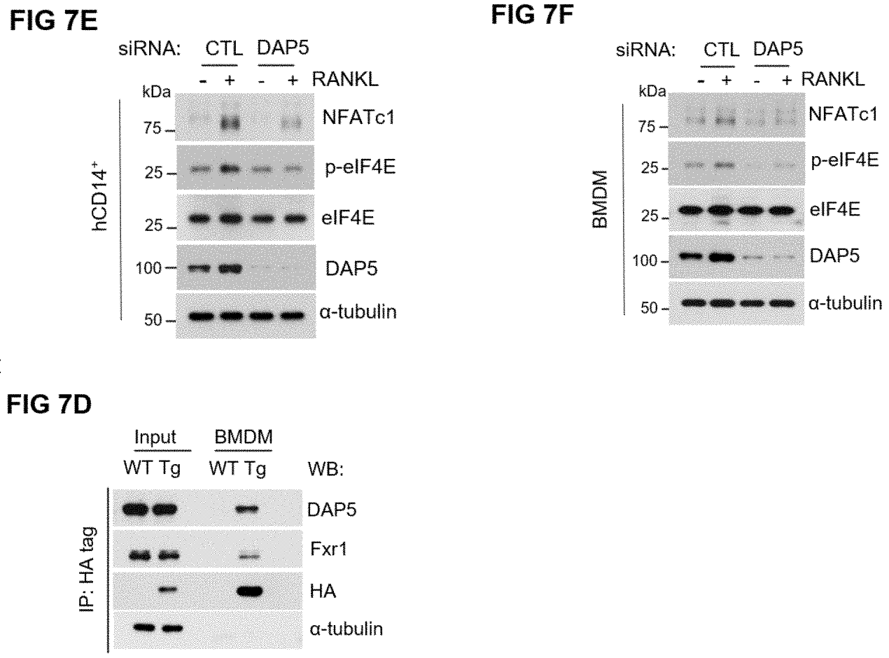

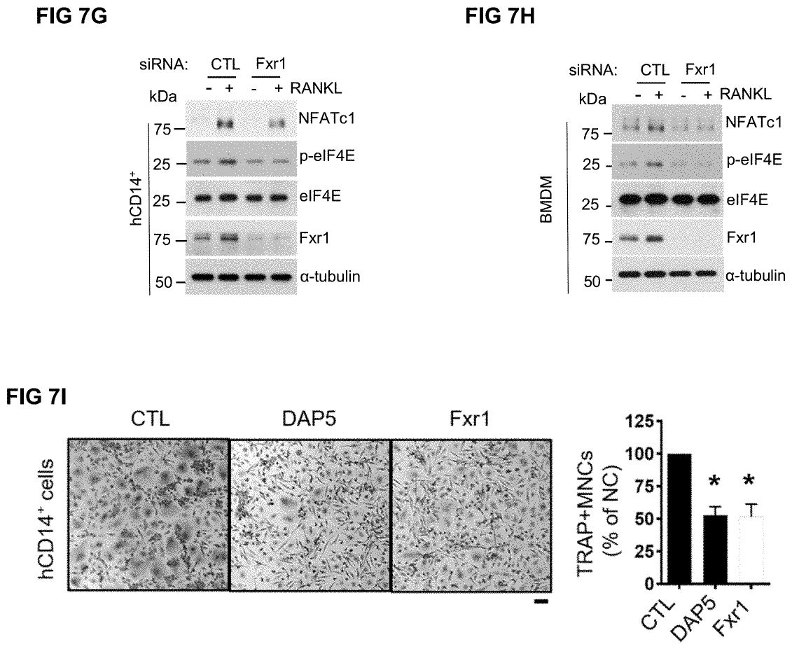

Ingenuity Pathway Analysis showed that 20 FICD-interacting proteins had enriched protein synthesis and post-transcriptional modifications (FIG. 7A). Among them, we focused on DAP5, which binds to MNK1 and belongs to protein translation initiation complexes (Pyronnet, S. et al. Human eukaryotic translation initiation factor 4G (eIF4G) recruits mnk1 to phosphorylate eIF4E. The EMBO journal 18, 270-279, doi:10.1093/emboj/18.1.270 (1999).) (FIG. 7, B and C). To corroborate the interaction between FICD and DAP5, we performed immunoprecipitation analysis using BMDMs of wild type and FICDtg^{M} mice. We detected that FICD bound to DAP5 (FIG. 7D). As Fxr1 was shown to form a complex with DAP5 34, we also tested if Fxr1 interacted with FICD. FICD also bound to Fxr1 (FIG. 7D), suggesting that FICD might interact with the DAP5/Fxr1 complex.

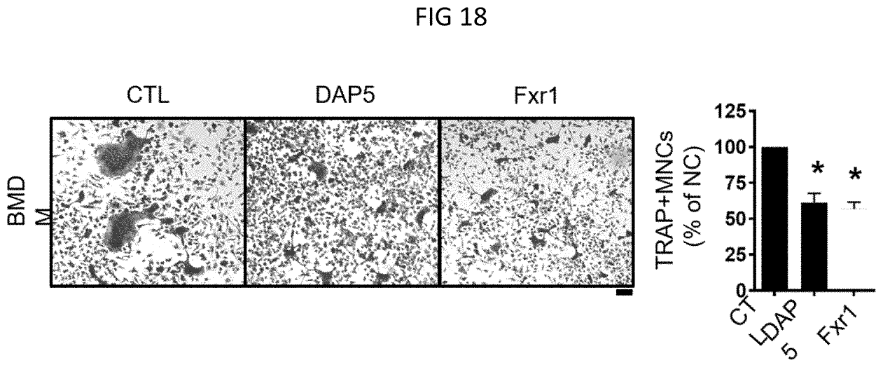

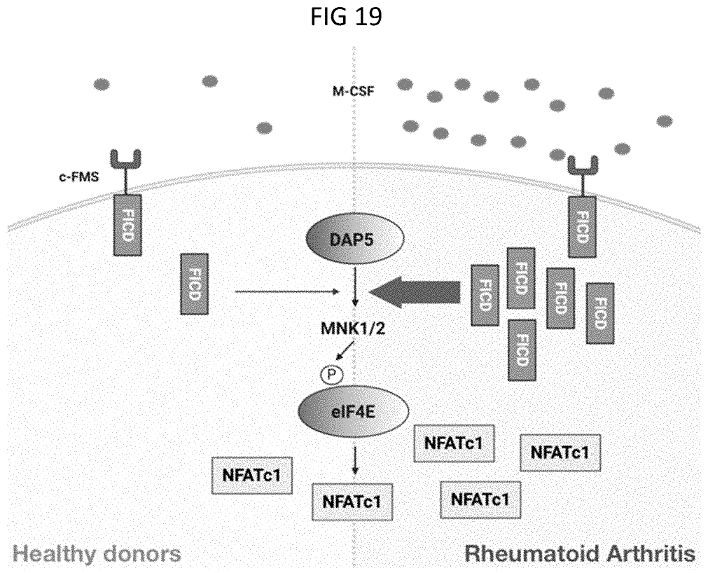

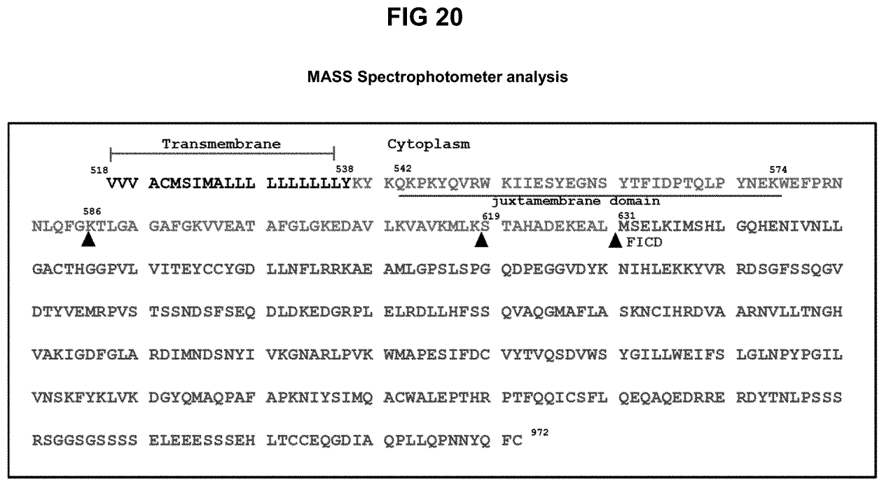

As the function of the DAP5/Fxr1 complex in OCPs has not been previously characterized, we tested the effect of the DAP5/Fxr1 complex on osteoclastogenesis by knocking down these proteins in both human and mouse osteoclasts using siRNAs. Both DAP5 and Fxr1 increased upon RANKL stimulation, and a knock down of DAP5 and Fxr1 suppressed their expression in both human and mouse OCPs (FIG. 7, E-H). Strikingly, the DAP5/Fxr1 deficiency suppressed RANKL-induced eIF4E phosphorylation and the expression of NFATc1 protein (FIG. 7, E-H), suggesting that the FICD/DAP5/Fxr1 axis plays an important role in eIF4E phosphorylation and NFATc1 expression in osteoclasts. Accordingly, osteoclast differentiation was also suppressed by DAP5 or Fxr1-deficiency (FIG. 7I and FIG. 18). Our data suggest that the DAP5/Fxr1 complex contributes to the activation of MMNK1/2/eIF4E and NFATc1 expression in osteoclasts, and also serves as a positive regulator of osteoclast differentiation. Taken together, our findings support that FICD promotes osteoclast differentiation by permitting the sustained activation of MNK1/2 and eIF4E phosphorylation, and in turn, NFATc1 expression is increased in FICDtg^{M} osteoclasts (FIG. 19).

### Example 3: Discussion

Cell surface receptors sense environmental stimuli and control cellular responses by activating downstream signaling cascades. However, recent studies revealed that the intramembrane cleavage of cell surface receptors also plays an important role in signaling processes and regulates cellular function. Here, we demonstrated that c-FMS proteolysis was critically involved in the osteoclastogenic responses of OCPs to the TNF family cytokine RANKL, and works cooperatively with the conventional M-CSF/c-FMS signaling pathways. c-FMS is processed into smaller intracellular fragments (FICDs) in OCPs by engaging c-FMS-mediated signaling pathways. FICDs formed a complex with DAP5 and activated the MNK1/2-eIF4E axis to enhance NFATc1 protein expression and osteoclastogenesis. Our data established FICD as a positive regulator of osteoclastogenesis. Furthermore, by modeling the increased FICDs in RA OCPs, myeloid cell-specific FICD expression enhanced in vivo osteoclastogenesis and promoted arthritic bone erosion in a murine arthritis model. These findings identify a novel function of c-FMS proteolysis in regulating (patho)physiological bone erosion and sensitivity to cytokine RANKL stimulation.

The altered expression of M-CSF and c-FMS have been implicated in the exacerbation of various diseases (Mun, S. H., Park, P. S. U. & Park-Min, K. H. The M-CSF receptor in osteoclasts and beyond. Exp Mol Med 52, 1239-1254, doi:10.1038/s12276-020-0484-z (2020).). To readjust the c-FMS-M-CSF/IL-34 axis, several drug discovery programs were aimed at finding inhibitors of the tyrosine kinase activity of c-FMS (Hamilton, J. A., Cook, A. D. & Tak, P. P. Anti-colony-stimulating factor therapies for inflammatory and autoimmune diseases. Nat Rev Drug Discov 16, 53-70, doi:10.1038/nrd.2016.231 (2016).). Although inhibiting c-FMS kinase activity appears to be an attractive strategy and has already shown promise, the prolonged use of c-FMS inhibitors is limited by their side effects. Targeting osteoclasts using denosumab, an anti-RANKL antibody, shows efficacy on the progression of arthritic bone erosion without affecting RA disease activity (Ishiguro, N. et al. Efficacy of denosumab with regard to bone destruction in prognostic subgroups of Japanese rheumatoid arthritis patients from the phase II DRIVE study. Rheumatology (Oxford) 58, 997-1005, doi:10.1093/rheumatology/key416 (2019). Cohen, S. B. et al. Denosumab treatment effects on structural damage, bone mineral density, and bone turnover in rheumatoid arthritis: a twelve-month, multicenter, randomized, double-blind, placebo-controlled, phase II clinical trial. Arthritis Rheum 58, 1299-1309, doi:10.1002/art.23417 (2008).), emphasizing the importance of osteoclasts in arthritic bone erosion. A better understanding of osteoclast regulation in arthritis is important for developing osteoclast-specific therapeutic interventions for arthritic bone erosion. We demonstrated that FICD overexpression using transgenic FICD knock-in mice affected osteoclasts with no effect on disease activity, while inhibiting c-FMS signals attenuated both disease activity and arthritic bone erosion in murine arthritis models. This is consistent with our observations that blocking c-FMS proteolysis had no effect on inflammation. In normal macrophages, the FICD level was very low. However, high FICD expression was found in RA synovial CD14+ cells which have a higher potential to differentiate into osteoclasts. Many plausible causes for arthritic bone erosion have been identified. Our study revealed the pathophysiological importance of FICD and its associated pathways in arthritic bone erosion and suggests that inhibiting FICD generation or function in RA patients who have high FICD expression in OCPs might be beneficial for inflammatory bone destruction.

Our results demonstrated that c-FMS proteolysis is not only involved in protein turnover but also in generating the necessary functional elements to promote osteoclastogenesis. High levels of M-CSF in RA synovium and RA synovial fluids may contribute to c-FMS proteolysis and generating FICDs. c-FMS proteolysis has been considered a disposal mechanism, which is coupled with proteosomal degradation of c-FMS. When cells were exposed to inflammatory mediators, c-FMS proteolysis started immediately, and c-FMS rapidly degraded (Carlberg, K., Tapley, P., Haystead, C. & Rohrschneider, L. The role of kinase activity and the kinase insert region in ligand-induced internalization and degradation of the c-fms protein. The EMBO journal 10, 877-883 (1991).). Inhibiting proteosomal degradation with bortezomib suppresses osteoclastogenesis by promoting c-FMS degradation Terpos, E., Sezer, O., Croucher, P. & Dimopoulos, M. A. Myeloma bone disease and proteasome inhibition therapies. Blood 110, 1098-1104, doi:10.1182/blood-2007-03-067710 (2007). Lee, K. et al. Blocking of the Ubiquitin-Proteasome System Prevents Inflammation-Induced Bone Loss by Accelerating M-CSF Receptor c-Fms Degradation in Osteoclast Differentiation. International journal of molecular sciences 18, doi:10.3390/ijms18102054 (2017)).

Our data showed that c-FMS-mediated signals are required for FICD generation. FMSmut does not generate FICDs and exhibits impaired osteoclastogenesis. Although we demonstrated that FMSmut is a functional receptor, we could not exclude that FMSmut may affect other signaling pathways that play an important role in osteoclastogenesis. However, our data from FICDtg^{M} mice support that the impaired FICD generation in FMSmut is likely to affect osteoclastogenesis. Our study extended the current paradigm of the c-FMS signaling network by demonstrating that c-FMS proteolysis is a new player in the c-FMS signaling network.

The MNK1/2/p-eIF4e axis is downstream of mediators of FICDs and interact with DAP5/Fxr1 complexes. The role of DAP5 and Fxr1 in osteoclasts has not been explored. We showed that DAP5 or Fxr1 deficiency suppressed NFATc1 expression and osteoclastogenesis. However, FICD/DAP5/Fxr1 complexes can target other proteins in addition to NFATc1 to suppress osteoclastogenesis. Further investigation of the mechanisms by which c-FMS proteolysis regulates the function of DAP5 is needed for a deeper understanding. Moreover, inhibiting MNK1/2 activity suppressed osteoclastogenesis and arthritic bone erosion and our study provides important insights into the FICD/DAP5/Fxr1/MNK1/2 axis' amenability to therapeutic intervention. Overall, FICD activity on osteoclast differentiation and bone resorption under pathological conditions can be determined by integrating the M-CSF levels, effect of proteases, and FICD interacting proteins.

### Sequence Listing Free Text:

### SEQ ID Nos 1-33 <213> Artificial Sequence <223> Primer

All publications cited in this specification are incorporated herein by reference. In addition, U.S. Provisional Patent Application No. 62/902,782, filed Sep. 19, 2019, is incorporated herein by reference. While the invention has been described with reference to particular embodiments, it will be appreciated that modifications can be made without departing from the spirit of the invention. Such modifications are intended to fall within the scope of the appended claims.

1. A method of treating bone resorption associated with osteoclastic activity in a subject in need thereof, comprising reducing the level of FMS intracellular fragments (FICDs) in the subject.

2. The method of claim 1, wherein the FICDs are located in human synovial CD 14+ cells.

3. The method of claim 1, comprising administering an inhibitor of MNK1/2.

4. The method of claim 3, wherein the MNK1/2 inhibitor is an MNK1, MNK2, or pan-MNK inhibitor.

5. The method of claim 3, wherein the MNK1/2 inhibitor is selected from CGP 57380, timovosertib (eFT-508), ETC-206, SLV-2436, and cercosporamide.

6. The method of claim 1, comprising administering an inhibitor of calpain 1 or pan-Calpain inhibitor.

7. The method of claim 6, wherein the calpain 1 inhibitor is selected from BDA-410, PD 151746, ALLM, MDL-28170, calpeptin, ALLN, PD 150606, calpain inhibitor XII, Z-L-Abu-CONH-ethyl, and Z-L-Abu-CONH(CH2)3-morpholine.

8. The method of claim 1, comprising inhibiting TNF-alpha converting enzyme (TACE).

9. The method of claim 8, comprising administering a blocking peptide comprising the TACE cleavage site of c-FMS.

10. The method of claim 9, wherein the blocking peptide has a sequence comprising LGQSKQ with up to 3 amino acid substitutions.

11. The method of claim 1, wherein the subject has rheumatoid arthritis, bone metastasis, periodontitis, osteoporosis or osteopenia.

12. A method of diagnosing and treating bone loss associated with osteoclastic activity in a subject, the method comprising:
(i) quantifying the amount of FMS intracellular fragments (FICDs) in a sample from the subject; and/ or
(ii) quantifying the amount of circulating soluble c-FMSFICDs in a sample from the subject; and
(iii) diagnosing a bone loss in the subject when an increase in FICDs or soluble c-FMS is detected as compared to a control; and
(iv) treating the subject for bone loss.

13. The method according to claim 12, wherein the subject is diagnosed with rheumatoid arthritis, osteoporosis, or osteopenia.

14. (canceled)

15. The method according to claim 12, wherein said FICDs are approximately 50 kD (H-FICD) and/or 48 kD (L-FICD).

16. The method according to claim 12, wherein said FICDs are detected via antibodies directed to the C-terminus of c-FMS.

17. The method of claim 12, wherein the subject is treated for bone loss using antiresorptive therapy or a Disease-Modifying Drug (DMARD).

18. A method of assessing the efficacy of a treatment for a bone loss, the method comprising:
(i) quantifying the amount of FICDs in a sample from the subject; or
(ii) quantifying the amount of soluble c-FMS in a sample from the subject; wherein a decrease in the amount of the amount of circulating FICDs or soluble c-FMS as compared to a control indicates the treatment is at least partially efficacious.

19. The method according to claim 18, wherein the treatment is a bisphosphonate or Disease-Modifying Drug (DMARD).

20. The method according to claim 12, wherein the control is an FICD or soluble c-FMS level obtained from the subject at an earlier time point.

21. The method according to claim 12, wherein the control is an FICD or soluble c-FMS level obtained from a healthy subject or healthy population of subjects.


SEQUENCE LISTING (33 sequences)

SEQ ID NO: 1 - DNA, 20 bp, Artificial Sequence
  Primer
accctttcctgcgccccaga

SEQ ID NO: 2 - DNA, 20 bp, Artificial Sequence
  Primer
gttttggagctgctggcgcc

SEQ ID NO: 3 - DNA, 19 bp, Artificial Sequence
  Primer
tgccgtttgctgagtgtcc

SEQ ID NO: 4 - DNA, 20 bp, Artificial Sequence
  Primer
tctcctccgacatcctcggg

SEQ ID NO: 5 - DNA, 16 bp, Artificial Sequence
  Primer
ctcggccggtgttccc

SEQ ID NO: 6 - DNA, 20 bp, Artificial Sequence
  Primer
ccggcgtgcccttatagtag

SEQ ID NO: 7 - DNA, 21 bp, Artificial Sequence
  Primer
gctgttccattgagtctccca

SEQ ID NO: 8 - DNA, 20 bp, Artificial Sequence
  Primer
gggtttctcaggcgaaccat

SEQ ID NO: 9 - DNA, 22 bp, Artificial Sequence
  Primer
cttcttccagtattccacctat

SEQ ID NO: 10 - DNA, 22 bp, Artificial Sequence
  Primer
ttgccctaattacctgttgaag

SEQ ID NO: 11 - DNA, 20 bp, Artificial Sequence
  Primer
gaccagtcaacaggggacat

SEQ ID NO: 12 - DNA, 20 bp, Artificial Sequence
  Primer
cctgaccaaggaaagcaaag

SEQ ID NO: 13 - DNA, 20 bp, Artificial Sequence
  Primer
tgtctacacggttcagagcg

SEQ ID NO: 14 - DNA, 20 bp, Artificial Sequence
  Primer
gggtagggattcagcccaag

SEQ ID NO: 15 - DNA, 20 bp, Artificial Sequence
  Primer
cccgtcacattctggtccat

SEQ ID NO: 16 - DNA, 20 bp, Artificial Sequence
  Primer
tctcctccgacatcctcggg

SEQ ID NO: 17 - DNA, 20 bp, Artificial Sequence
  Primer
tcctcagaccgctttttgcc

SEQ ID NO: 18 - DNA, 20 bp, Artificial Sequence
  Primer
ctaatcacgacgctgggact

SEQ ID NO: 19 - DNA, 20 bp, Artificial Sequence
  Primer
gtcaggttgcctctgtctca

SEQ ID NO: 20 - DNA, 20 bp, Artificial Sequence
  Primer
tcagggaagagtctggaaag

SEQ ID NO: 21 - DNA, 22 bp, Artificial Sequence
  Primer
aagccagagtccttcagagaga

SEQ ID NO: 22 - DNA, 20 bp, Artificial Sequence
  Primer
ggaaattggggtaggaagga

SEQ ID NO: 23 - DNA, 22 bp, Artificial Sequence
  Primer
ggtgcttgcctttatgccttta

SEQ ID NO: 24 - DNA, 21 bp, Artificial Sequence
  Primer
tggctgccatgaacaaaggtt

SEQ ID NO: 25 - DNA, 24 bp, Artificial Sequence
  Primer
caggtcgccatagcaacagtactc

SEQ ID NO: 26 - DNA, 25 bp, Artificial Sequence
  Primer
agtcgcagatctgcaagctaattcc

SEQ ID NO: 27 - DNA, 27 bp, Artificial Sequence
  Primer
gggccatttaccgtaagttatgtaacg

SEQ ID NO: 28 - DNA, 24 bp, Artificial Sequence
  Primer
gccatttaagccatgggaagttag

SEQ ID NO: 29 - DNA, 24 bp, Artificial Sequence
  Primer
tggacagaggagccataactgcag

SEQ ID NO: 30 - DNA, 24 bp, Artificial Sequence
  Primer
ggtacaggctcccagaaggttgac

SEQ ID NO: 31 - DNA, 25 bp, Artificial Sequence
  Primer
caacgtgctggttattgtgctgtct

SEQ ID NO: 32 - DNA, 27 bp, Artificial Sequence
  Primer
gggtctagaatgtccgagctgaagatc

SEQ ID NO: 33 - DNA, 26 bp, Artificial Sequence
  Primer
gggataccgactgcattaatgctgtt